SEQUENCE LISTING (41 sequences)

SEQ ID NO: 1 - protein, 17 aa, synthetic construct
  source 1..17: note = GM-CSF signal peptide
MWLQSLLLLGTVACSIS

SEQ ID NO: 2 - protein, 21 aa, synthetic construct
  source 1..21: note = CD8 alpha signal peptide
MALPVTALLLPLALLLHAARP

SEQ ID NO: 3 - protein, 47 aa, synthetic construct
  source 1..47: note = CD8 alpha hinge domain
KPTTTPAPRPPTPAPTIASQPLSLRPEACRPAAGGAVHTRGLDFACD

SEQ ID NO: 4 - protein, 24 aa, synthetic construct
  source 1..24: note = CD8 alpha transmembrane region
IYIWAPLAGTCGVLLLSLVITLYC

SEQ ID NO: 5 - protein, 42 aa, synthetic construct
  source 1..42: note = 4-1BB co-stimulatory signaling domain
KRGRKKLLYIFKQPFMRPVQTTQEEDGCSCRFPEEEEGGCEL

SEQ ID NO: 6 - protein, 21 aa, synthetic construct
  source 1..21: note = DAP10 intracellular signaling domain
RPRRSPAQEDGKVYINMPGRG

SEQ ID NO: 7 - protein, 113 aa, synthetic construct
  source 1..113: note = DAP12
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 8 - protein, 112 aa, synthetic construct
  source 1..112: note = CD3 zeta primary intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 14 aa, synthetic construct
  source 1..14: note = KIR2DS2 hinge
SPTEPSSKTGNPRH

SEQ ID NO: 10 - protein, 22 aa, synthetic construct
  source 1..22: note = KIR2DS2 transmembrane domain
LHVLIGTSVVKIPFTILLFFLL

SEQ ID NO: 11 - protein, 39 aa, synthetic construct
  source 1..39: note = KIR2DS2 intracellular signaling domain
HRWCSNKKNAAVMDQEPAGNRTVNSEDSDEQDHQEVSYA

SEQ ID NO: 12 - protein, 40 aa, synthetic construct
  source 1..40: note = Furin-V5-SGSG-P2A
KRRGKPIPNPLLGLDSTSGSGATNFSLLKQAGDVEENPGP

SEQ ID NO: 13 - protein, 241 aa, synthetic construct
  source 1..241: note = BCMA VHH1-VHH2
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 14 - protein, 162 aa, synthetic construct
  source 1..162: note = IL-15 Preprotein sequence
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 15 - protein, 508 aa, synthetic construct
  source 1..508: note = 31 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRPRRSPAQEDGKVYINMPGRG

SEQ ID NO: 16 - protein, 536 aa, synthetic construct
  source 1..536: note = 32 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRLVPRGRGAAEAATRKQRITETESPYQELQGQRSDVYSDLNTQRPYYK

SEQ ID NO: 17 - protein, 500 aa, synthetic construct
  source 1..500: note = 36 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSGGGGSGGGGSSPTEPSSKTGNPRHLHVLIGTSVVKIPFTILLFFLLHRWCSNKKNAAVMDQEPAGNRTVNSEDSDEQDHQEVSYAKRRGKPIPNPLLGLDSTSGSGATNFSLLKQAGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 18 - protein, 484 aa, synthetic construct
  source 1..484: note = 37 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHRWCSNKKNAAVMDQEPAGNRTVNSEDSDEQDHQEVSYA

SEQ ID NO: 19 - protein, 637 aa, synthetic construct
  source 1..637: note = 38 CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHRWCSNKKNAAVMDQEPAGNRTVNSEDSDEQDHQEVSYAKRRGKPIPNPLLGLDSTSGSGATNFSLLKQAGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 20 - protein, 487 aa, synthetic construct
  source 1..487: note = BBZ CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 486 aa, synthetic construct
  source 1..486: note = CD28Z CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSKPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 539 aa, synthetic construct
  source 1..539: note = 352M CAR
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCYFLGRLVPRGRGAAEAATRKQRITETESPYQELQGQRSDVYSDLNTQRPYYKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 119 aa, synthetic construct
  source 1..119: note = First VHH BCMA 269A37948
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 24 - protein, 117 aa, synthetic construct
  source 1..117: note = Second VHH BCMA 269AS34822
QVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 25 - protein, 5 aa, synthetic construct
  source 1..5: note = CDR1 of the first anti-BCMA VHH antibody moiety
TYFMA

SEQ ID NO: 26 - protein, 17 aa, synthetic construct
  source 1..17: note = CDR2 of the first anti-BCMA VHH antibody moiety
GIAWSGGSTAYADSVKG

SEQ ID NO: 27 - protein, 10 aa, synthetic construct
  source 1..10: note = CDR3 of the first anti-BCMA VHH antibody moiety
RGIEVEEFGA

SEQ ID NO: 28 - protein, 5 aa, synthetic construct
  source 1..5: note = CDR1 of the second anti-BCMA VHH antibody moiety
NYYMG

SEQ ID NO: 29 - protein, 16 aa, synthetic construct
  source 1..16: note = CDR2 of the second anti-BCMA VHH antibody moiety
IISSDTTITYKDAVKG

SEQ ID NO: 30 - protein, 9 aa, synthetic construct
  source 1..9: note = CDR3 of the second anti-BCMA VHH antibody moiety
WTSDWSVAY

SEQ ID NO: 31 - protein, 114 aa, synthetic construct
  source 1..114: note = IL-15 polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 49 aa, synthetic construct
  source 1..49: note = Intracellular domain of DAP12
GRLVPRGRGAAEAATRKQRITETESPYQELQGQRSDVYSDLNTQRPYYK

SEQ ID NO: 33 - protein, 267 aa, synthetic construct
  source 1..267: note = CD8 alpha Hinge-CD8 alpha TM-CD137 CD3 zeta DAP10 ICD
MALPVTALLLPLALLLHAARPKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRPRRSPAQEDGKVYINMPGRG

SEQ ID NO: 34 - protein, 295 aa, synthetic construct
  source 1..295: note = CD8 alpha Hinge-CD8 alpha TM-CD137 CD3 zeta DAP12 ICD
MALPVTALLLPLALLLHAARPKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRLVPRGRGAAEAATRKQRITETESPYQELQGQRSDVYSDLNTQRPYYK

SEQ ID NO: 35 - protein, 259 aa, synthetic construct
  source 1..259: note = KIR2DS2 Hinge-KIR2DS2 TM-KIR2DS2 DAP12 ICD
MALPVTALLLPLALLLHAARPGGGGSGGGGSSPTEPSSKTGNPRHLHVLIGTSVVKIPFTILLFFLLHRWCSNKKNAAVMDQEPAGNRTVNSEDSDEQDHQEVSYAKRRGKPIPNPLLGLDSTSGSGATNFSLLKQAGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 36 - protein, 243 aa, synthetic construct
  source 1..243: note = CD8 alpha Hinge-CD8 alpha TM-CD3 zeta KIR2DS2 ICD
MALPVTALLLPLALLLHAARPKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHRWCSNKKNAAVMDQEPAGNRTVNSEDSDEQDHQEVSYA

SEQ ID NO: 37 - protein, 396 aa, synthetic construct
  source 1..396: note = CD8 alpha Hinge-CD8 alpha TM-CD3 zeta KIR2DS2 DAP12 ICD
MALPVTALLLPLALLLHAARPKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHRWCSNKKNAAVMDQEPAGNRTVNSEDSDEQDHQEVSYAKRRGKPIPNPLLGLDSTSGSGATNFSLLKQAGDVEENPGPMGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 38 - protein, 5 aa, synthetic construct
  source 1..5: note = Linker sequence
GGGGS

SEQ ID NO: 39 - protein, 15 aa, synthetic construct
  source 1..15: note = Linker sequence
GGGGSGGGGSGGGGS

SEQ ID NO: 40 - protein, 22 aa, synthetic construct
  source 1..22: note = P2A
GSGATNFSLLKQAGDVEENPGP

SEQ ID NO: 41 - protein, 45 aa, synthetic construct
  source 1..45: note = CD8 alpha hinge domain 1
TTTPAPRPPTPAPTIASQPLSLRPEACRPAAGGAVHTRGLDFACD

(19) United States
(12) Patent Application Publication
(10) Pub. No.: US 2026/0325635 A1
(43) Pub. Date: Oct. 22, 2026

(54) CHIMERIC ANTIGEN RECEPTORS AND USES THEREOF

(71) Applicant: Legend Biotech USA Inc., Somerset, NJ (US)

(72) Inventors: Tianjian LI, Somerset, NJ (US); Rohit Mathur, Somerset, NJ (US); Ming Zeng, Nanjing (CN); Huihui ZHANG, Nanjing (CN)

(21) Appl. No.: 19/476,566

(22) PCT Filed: May 24, 2024
(86) PCT No.: PCT/CN2024/095081

§ 371 Date: Oct. 17, 2025

(30) Foreign Application Priority Data
May 25, 2023 (WO) PCT/CN2023/096342

(51) Int. Cl. A61K 40/31; A61K 35/17; A61K 40/15; A61K 40/42; A61P 35/00; C07K 14/54; C07K 14/705; C07K 16/28; C12N 5/0783

(52) U.S. Cl. CPC A61K 40/31; A61K 35/17; A61K 40/15; A61K 40/4203; A61P 35/00; C07K 14/5443; C07K 14/705; C07K 14/70503; C07K 14/7051; C07K 14/70517; C07K 14/70596; C07K 16/2878; C12N 5/0646; A61K 2239/13; A61K 2239/17; A61K 2239/21; A61K 2239/22; C07K 2317/565; C07K 2317/569; C12N 2510/00

(57) ABSTRACT

Provided herein are chimeric antigen receptors (CARs) and cells (e.g., natural killer cells) expressing the CARs.

### CROSS REFERENCE TO RELATED APPLICATION

This application claims benefit of priority of International Patent Application No. PCT/CN2023/096342 filed on May 25, 2023, the content of which is incorporated herein by reference in its entirety.

### SEQUENCE STATEMENT

This application contains a Sequence Listing that has been submitted electronically as an XML file named “IEC240132PCT SEQUENCE LISTING.XML.” The XML file, created on May 24, 2024, is 50,848 bytes in size. The material in the XML file is hereby incorporated by reference in its entirety.

### TECHNICAL FIELD

The disclosure relates to chimeric antigen receptors (CARs) and cells (e.g., natural killer cells) expressing the CARs.

### BACKGROUND

Chimeric antigen receptor (CAR)-based therapies are a rapidly emerging form of cancer treatment, and have resulted in remarkable responses in diseases such as refractory lymphoid malignancies. However, their clinical use is limited by toxicity related to cytokine release syndrome and neurotoxicity, the logistic complexity of their manufacturing, cost and time-to-treatment, and the risk of graft-versus-host disease (GvHD) associated with the immune cell therapy.

Natural killer (NK) cells have emerged as a promising source of cells for CAR-based therapies due to their ready availability and safety profile. Since the activation process of NK cells is different from T cells, there exists a need for improved CAR designs with NK-specific signaling domains for potent NK cell activation and effector functions.

### SUMMARY

Provided herein are chimeric antigen receptors (CARs) and cells (e.g., natural killer cells) expressing the CARs.

In one aspect, the present disclosure is related to a chimeric antigen receptor (CAR) comprising an extracellular antigen-binding domain; a transmembrane region; and an intracellular domain comprising a DAP10 intracellular signaling domain, a DAP12 intracellular signaling domain or DAP12, or a KIR2DS2 intracellular signaling domain.

In some embodiments, the intracellular domain further comprises an activation domain comprising a CD3ζ primary intracellular signaling domain.

In some embodiments, the DAP10 intracellular signaling domain, the DAP12 intracellular signaling domain, or the KIR2DS2 intracellular signaling domain is located at the C-terminus of the intracellular domain.

In some embodiments, the CAR further comprises a co-stimulatory signaling domain located between the C-terminus of the transmembrane region and the N-terminus of the CD3ζ primary intracellular signaling domain.

In some embodiments, the co-stimulatory signaling domain is CD137 (4-1BB) co-stimulatory signaling domain.

In some embodiments, the intracellular domain comprises a KIR2DS2 intracellular signaling domain.

In some embodiments, the CAR further comprises a DAP12 at the C-terminus of the intracellular signaling domain.

In some embodiments, the transmembrane region is a transmembrane region derived from: α chain of a T cell receptor, β chain of the T cell receptor, ζ chain of the T cell receptor, CD8α, CD28, CD3ε, CD3δ, CD3γ, CD33, CD37, CD64, CD80, CD45, CD4, CD5, CD8, CD9, CD16, CD22, CD86, CD137 or CD154.

In some embodiments, the transmembrane region is a transmembrane region derived from CD8α.

In some embodiments, the CAR further comprises a hinge domain located between the C-terminus of the extracellular antigen binding domain and the N-terminus of the transmembrane domain.

In some embodiments, the hinge region is a hinge region derived from: CD8α, CD28, IgG1, IgG2, IgG3, or IgG4.

In some embodiments, the hinge region is a hinge region derived from CD8α.

In some embodiments, the CAR comprises from the N-terminus to the C-terminus as following: an extracellular antigen-binding domain; a CD8α hinge region; a CD8α transmembrane region; and a CD137 (4-1BB) co-stimulatory signaling domain, a CD3ζ primary intracellular signaling domain, and a DAP12 intracellular signaling domain.

In some embodiments, the transmembrane region is a transmembrane region derived from KIR2DS2.

In some embodiments, the CAR further comprise a hinge domain located between the C-terminus of the extracellular antigen binding domain and the N-terminus of the transmembrane domain, wherein the hinge is a hinge region derived from KIR2DS2.

In some embodiments, the intracellular domain comprises a KIR2DS2 intracellular signaling domain.

In some embodiments, the CAR further comprises a DAP12 at the C-terminus of the intracellular signaling domain.

In some embodiments, the extracellular antigen-binding domain comprises a scFv, a (scFv)2, a VHH domain, or a VNAR domain.

In some embodiments, the extracellular antigen-binding domain comprises one or more VHH domains.

In some embodiments, the extracellular antigen-binding domain binds to a tumor antigen.

In some embodiments, the tumor antigen is selected from the group consisting of: B-cell maturation antigen (BCMA), CD34, CD45, human leukocyte antigen-DR (HLA-DR), CD123, CD38, CLL1, CD105, CD71, SSC, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125, CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-13, IL-6, insulin-like growth factor I receptor, integrin a5b1, integrin anb3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-R1, TRAIL-R2, tumor antigen CTAA16.88, VEGF-A, VEGFR-1, VEGFR2, and vimentin.

In some embodiments, the extracellular antigen-binding domain binds to BCMA.

In some embodiments, the CAR comprises a first VHH antibody moiety that comprises a CDR1 comprising the amino acid sequence of SEQ ID NO: 25, a CDR2 comprising the amino acid sequence of SEQ ID NO: 26, and a CDR3 comprising the amino acid sequence of SEQ ID NO: 27, and a second VHH antibody moiety that comprises a CDR1 comprising the amino acid sequence of SEQ ID NO: 28, a CDR2 comprising the amino acid sequence of SEQ ID NO: 29, and a CDR3 comprising the amino acid sequence of SEQ ID NO: 30.

In some embodiments, the CAR comprises a first VHH antibody moiety comprising the amino acid sequences of SEQ ID NO: 23 or an amino acid sequence having at least 90%, 95%, or 99% identity to the amino acid sequence of SEQ ID NO: 23, and a second VHH antibody moiety comprising the amino acid sequences of SEQ ID NO: 24 or an amino acid sequence having at least 90%, 95%, or 99% identity to the amino acid sequence of SEQ ID NO: 24.

In some embodiments, the CAR comprises an amino acid sequence set forth in SEQ ID NO: 13, or an amino acid sequence having at least 90%, 95%, or 99% identity to the amino acid sequence set forth in any one of SEQ ID NO: 13.

In some embodiments, the CAR comprises an amino acid sequence of any one of SEQ ID NOs: 15-16 or an amino acid sequence that is at least 80%, 85%, 90% or 95% identical to any one of SEQ ID NOs: 15-16.

In some embodiments, the CAR comprises an amino acid sequence of SEQ ID NO: 18 or an amino acid sequence that is at least 80%, 85%, 90% or 95% identical to SEQ ID NO: 18.

In some embodiments, the CAR comprises an amino acid sequence of SEQ ID NO: 19 or an amino acid sequence that is at least 80%, 85%, 90% or 95% identical to SEQ ID NO: 19.

In some embodiments, the CAR comprises an amino acid sequence of SEQ ID NO: 17 or an amino acid sequence that is at least 80%, 85%, 90% or 95% identical to SEQ ID NO: 17.

In one aspect, the present disclosure is related to a nucleotide sequence encoding the CAR described herein.

In one aspect, the present disclosure is related to a modified immune cell comprising the nucleic acid described herein.

In some embodiments, the modified immune cell is selected from the group consisting of: a natural killer (NK) cell, T cell, a B cell, and a macrophage/monocyte, peripheral blood mononuclear cell (PBMC), hematopoietic stem cell, pluripotent stem cell, and an embryonic stem cell.

In some embodiments, the cell is NK cell.

In some embodiments, the cell further comprises a nucleotide sequence encoding an IL-15 polypeptide.

In some embodiments, the IL-15 polypeptide comprises an amino acid substitution at position 62.

In some embodiments, the amino acid substitution at position 62 is T62G.

In some embodiments, the IL-15 polypeptide comprises an amino acid sequence having at least 90% sequence identity to the amino acid sequence of SEQ ID NO: 31.

In one aspect, the present disclosure is related to a pharmaceutical composition comprising the modified immune cell described herein, and a pharmaceutical acceptable carrier.

In one aspect, the present disclosure is related to a method for treating and/or preventing cancer comprising administering an effective amount of the modified immune cell described herein or pharmaceutical composition described herein to a subject in need thereof.

As used herein, the terms “intracellular domain”, “intracellular region” or “cytoplasmic region” are used interchangeably herein to refer to the portion of a receptor that is inside the cell. The intracellular domain can be the entire portion of a receptor that is inside the cell, or just a part thereof (e.g., at least 50%, 60%, 70%, 80%, 85%, 90%, 95%, 96%, 97%, 98%, or 99% of the entire portion). The intracellular domain can be derived from the intracellular domain of a wild-type membrane protein (e.g., a receptor) or a functional variant thereof. In some embodiments, the intracellular domain can have one or more mutations, including e.g., insertions, deletions, and/or substitutions.

As used herein, the term “DAP10 intracellular signaling domain” refers to the intracellular signaling domain derived from DAP10 or a functional variant thereof. In some embodiments, the DAP10 intracellular signaling domain can have one or more mutations, including e.g., insertions, deletions, and/or substitutions. In some embodiments, the DAP10 intracellular signaling domain can comprise a sequence of SEQ ID NO: 6 or a sequence that is at least 80%, 85%, 90%, or 95% identical to SEQ ID NO: 6.

As used herein, the term “DAP12 intracellular signaling domain” refers to the intracellular signaling domain derived from DAP12 or a functional variant thereof. In some embodiments, the DAP12 intracellular signaling domain can have one or more mutations, including e.g., insertions, deletions, and/or substitutions. In some embodiments, the DAP12 intracellular signaling domain can comprise a sequence of SEQ ID NO: 32 or a sequence that is at least 80%, 85%, 90%, or 95% identical to SEQ ID NO: 32.

As used herein, the term “KIR2DS2 intracellular signaling domain” refers to the intracellular signaling domain derived from KIR2DS2 or a functional variant thereof. In some embodiments, the KIR2DS2 intracellular signaling domain can have one or more mutations, including e.g., insertions, deletions, and/or substitutions. In some embodiments, the KIR2DS2 intracellular signaling domain can comprise a sequence of SEQ ID NO: 11 or a sequence that is at least 80%, 85%, 90%, or 95% identical to SEQ ID NO: 11.

As used herein, the term “co-stimulatory signaling domain” refers to the portion of the receptor that includes one or more chimeric domains (e.g., endodomains) from co-stimulatory proteins for the receptor to persist after activation. The co-stimulatory signaling domain can be derived from the endodomains of a wild-type co-stimulatory protein or a functional variant thereof. In some embodiments, the co-stimulatory signaling domain can have one or more mutations, including e.g., insertions, deletions, and/or substitutions.

As used herein, the term “CD137 (4-1BB) co-stimulatory signaling domain” refers to the endodomains derived from CD137 (4-1BB) or a functional variant thereof. In some embodiments, the CD137 (4-1BB) co-stimulatory signaling domain can have one or more mutations, including e.g., insertions, deletions, and/or substitutions. In some embodiments, the CD137 (4-1BB) co-stimulatory signaling domain can comprise a sequence of SEQ ID NO: 5 or a sequence that is at least 80%, 85%, 90%, or 95% identical to SEQ ID NO: 5.

As used herein, the term “DAP12” refers to wildtype DAP12 or a functional variant thereof. In some embodiments, the DAP12 can have one or more mutations, including e.g., insertions, deletions, and/or substitutions. In some embodiments, the DAP12 can comprise a sequence of SEQ ID NO: 7 or a sequence that is at least 80%, 85%, 90%, or 95% identical to SEQ ID NO: 7.

As used herein, the term “IL-15” refers to wildtype IL-15 or a functional variant thereof (e.g., an IL-15 preprotein). In some embodiments, the IL-15 can have one or more mutations, including e.g., insertions, deletions, and/or substitutions. In some embodiments, the IL-15 can comprise a sequence of SEQ ID NO: 14 or 31 or a sequence that is at least 80%, 85%, 90%, or 95% identical to SEQ ID NO: 14 or 31. In some embodiments, the IL-15 polypeptide comprises an amino acid substitution at position 62, wherein the numbering of the amino acid residues corresponds to SEQ ID NO: 31. In some embodiments, the amino acid substitution at position 62 is T62G.

As used herein, the term “primary intracellular signaling domain” refers to cytoplasmic signaling sequence that acts in a stimulatory manner to induce immune effector functions. The primary intracellular signaling domain may contain a signaling motif known as immunoreceptor tyrosine-based activation motif, or ITAM. An “ITAM,” as used herein, is a conserved protein motif that is generally present in the tail portion of signaling molecules expressed in many immune cells. The motif can comprise two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs can also function as docking sites for other proteins involved in signaling pathways. Exemplary ITAM-containing primary cytoplasmic signaling sequences include those derived from CD3ζ, FcR gamma (FCERIG), FcR beta (Fc Epsilon Rib), CD3 gamma, CD3 delta, CD3 epsilon, CD5, CD22, CD79a, CD79b, and CD66d. In some embodiments, the primary intracellular signaling domain is derived from CD3ζ. In some embodiments, the intracellular signaling domain consists of a CD3ζ cytoplasmic signaling domain (e.g., SEQ ID NO: 8). In some embodiments, the primary intracellular signaling domain is derived from a cytoplasmic signaling domain of wildtype CD3. In some embodiments, the primary intracellular signaling domain is a functional mutant of the cytoplasmic signaling domain of CD3ζ containing one or more mutations, such as Q65K.

As used herein, the terms “transmembrane domain” or “transmembrane region” are used interchangeably herein to refer to the portion of a membrane protein (e.g., a receptor) that is embedded in the cell membrane. The transmembrane region can be entire portion of the protein that is embedded in the cell membrane, or just a part thereof (e.g., at least 50%, 60%, 70%, 80%, 85%, 90%, 95%, 96%, 97%, 98%, or 99% of the entire portion). The transmembrane region can be derived from the transmembrane region of a wild-type receptor or a functional variant thereof. In some embodiments, the transmembrane region can have one or more mutations, including e.g., insertions, deletions, and/or substitutions.

As used herein, the term “CD8α transmembrane region” refers to the transmembrane region derived from CD8α or a functional variant thereof. In some embodiments, the CD8α transmembrane region can have one or more mutations, including e.g., insertions, deletions, and/or substitutions. In some embodiments, the CD8α transmembrane region can comprise a sequence of SEQ ID NO: 4 or a sequence that is at least 80%, 85%, 90%, or 95% identical to SEQ ID NO: 4.

As used herein, the term “KIR2DS2 transmembrane region” refers to the transmembrane region derived from KIR2DS2 or a functional variant thereof. In some embodiments, the KIR2DS2 transmembrane region can have one or more mutations, including e.g., insertions, deletions, and/or substitutions. In some embodiments, the KIR2DS2 transmembrane region can comprise a sequence of SEQ ID NO: 10 or a sequence that is at least 80%, 85%, 90%, or 95% identical to SEQ ID NO: 10.

As used herein, the terms “hinge domain” or “hinge region” are used interchangeably herein to refer to the portion of a membrane protein (e.g., a receptor) that connects the transmarine region and the extracellular domain. In some embodiments, the hinge region can be part of an extracellular region. The hinge region can be derived from the hinge region of a wild-type receptor or a functional variant thereof. In some embodiments, the hinge region can have one or more mutations, including e.g., insertions, deletions, and/or substitutions.

As used herein, the term “CD8α hinge region” refers to the hinge region derived from CD8α or a functional variant thereof. In some embodiments, the CD8α hinge region can have one or more mutations, including e.g., insertions, deletions, and/or substitutions. In some embodiments, the CD8α hinge region can comprise a sequence of SEQ ID NO: 3 or 41 or a sequence that is at least 80%, 85%, 90%, or 95% identical to SEQ ID NO: 3 or 41.

As used herein, the term “KIR2DS2 hinge region refers to the hinge region derived from KIR2DS2 or a functional variant thereof. In some embodiments, the KIR2DS2 hinge region can have one or more mutations, including e.g., insertions, deletions, and/or substitutions. In some embodiments, the KIR2DS2 hinge region can comprise a sequence of SEQ ID NO: 9 or a sequence that is at least 80%, 85%, 90%, or 95% identical to SEQ ID NO: 9.

As used herein, the term “derived from” when made in reference to a domain described herein refers to a domain that is obtained from the relevant functional portion of a protein (e.g., by recombinant expression or de novo synthesis). The term encompasses domains with naturally occurring sequences and sequences with mutations. A domain derived from a particular protein can have a sequence that is at least 80%, 85%, 90%, 95%, 98%, 99%, or 100% identical to the relevant functional portion of the particular protein. A domain derived from a particular protein can be from a natural or a synthetic source.

As used herein, a “vector” is any construct capable of delivering one or more polynucleotides of interest to a host cell when the vector is introduced to the host cell. An “expression vector” is capable of delivering and expressing the one or more polynucleotides of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

As used herein, the term “chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular ligand binding domain or an extracellular antigen binding domain specific for one or more antigens (such as tumor antigens), a transmembrane domain, and an intracellular signaling domain. “CAR-T cell” refers to a T cell that expresses a CAR. “CAR-NK cell” refers to an NK cell that expresses a CAR.

As used herein, the term “cancer” or “cancer cell” refers to the cells dividing in an uncontrolled manner, e.g., forming the solid tumors or the excessive tumor cells in blood. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. The cancer cells can form the solid tumors or the excessive tumor cells in blood (e.g., hematologic cancer). Alternatively or additionally, it can include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Examples of cancers that can be treated by the methods described herein include e.g., bone cancer, pancreatic cancer, skin cancer (e.g., melanoma), cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia (AML), chronic myeloid leukemia, acute lymphoblastic leukemia, chronic lymphocytic leukemia, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, and/or T cell lymphoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1C show constructs evaluation using serial cytotoxicity assay. BCMA CAR-NK cells with different CAR constructs were evaluated for their cytotoxicity potential in a serial cytotoxicity assay. Co-cultures were set up with CAR-NK cells against NCI-H929 cells. FIG. 1A shows plan of repeat tumor addition, freshly labeled tumor cells were added to co-cultures in each round every 48 h. FIG. 1B shows long-term killing of various CAR-NK cells against H929 cells as evaluated up to seven rounds of repeat tumor addition. FIG. 1C shows percentage CAR during several rounds of serial cytotoxicity assay.

FIGS. 2A-2B show constructs evaluation using cytokine release assay. BCMA CAR-NK cells with different CAR constructs were evaluated for their cytokine release potential of IFN-γ (FIG. 2A) or GM-CSF (FIG. 2B) at 24 h post co-culture.

FIGS. 3A-3C show activity of selected CAR-NK cells in vivo. FIG. 3A shows in vivo cell-killing assay against luciferase labeled NCI-H929 cells bearing disseminated tumor mice model.

FIG. 3B shows PK/PD profile of human CAR positive NK cells. FIG. 3C shows PK/PD profile of CAR positive among total human cells.

FIGS. 4A-4D show activity of CAR-NK with new constructs in vitro. 32M CAR-NK better than BBzM CAR-NK, which is better than 352M CAR-NK. Newly designed BCMA CAR-NK cells with DAP12 at middle and early positions were evaluated. FIG. 4A shows plan of repeat tumor addition, freshly labeled tumor cells were added to co-cultures in each round every 24 h. FIG. 4B shows in vitro long term serial cytotoxicity assay against NCI-H929 cells. FIG. 4C shows percentage CAR positive NK cells during rounds of tumor exposure. FIG. 4D shows proliferation of CAR-NK cells during rounds of tumor exposure.

### DETAILED DESCRIPTION

Provided herein are chimeric antigen receptors (CARs) and cells (e.g., natural killer cells) expressing the CARs. The CARs described herein have superior NK cell activation efficiency and cytotoxicity on cancer cells. The CARs and immune cells (NK cells) expressing the CARs can be used in immune cell therapies with various cancer-targeting antibodies to treat different types of cancers.

### Chimeric Antigen Receptors (CARs)

A chimeric antigen receptor typically comprises an extracellular domain capable of binding to an antigen, and an intracellular domain comprising one or more intracellular signaling domains derived from signal transducing proteins. These intracellular signaling domains are typically different from the polypeptide from which the extracellular domain is derived. The extracellular domain can be any proteinaceous molecule or part thereof that can specifically bind to a predetermined antigen. The extracellular domain may comprise an antibody or antigen binding fragment thereof. The intracellular signaling domain can be any oligopeptide or polypeptide domain known to function to transmit a signal causing activation or inhibition of a biological process in a cell, for example, activation of an immune cell such as a T cell or a NK cell.

Chimeric antigen receptors (CARs) combine many facets of normal T cell or NK cell activation into a single protein. They link an extracellular antigen recognition domain to an intracellular signaling domain, which activates the T cell or NK cell when an antigen is bound. CARs typically have the following regions: an antigen binding domain, an extracellular hinge region, a transmembrane region, and an intracellular domain. The intracellular region may comprise an intracellular signaling domain or an intracellular signaling region.

A detailed review of CARs and CAR-expressing cells can be found in, e.g., J Exp Clin Cancer Res, 2022 Mar. 31; 41(1):119; Mol Cancer, 2023 Jan. 30; 22(1):20; and Br J Haematol, 2021 April; 193(2):216-230.doi: 10.1111/bjh.17186. Epub 2020 Nov. 20, each of which is incorporated by reference in its entirety. Exemplary structure of antigen receptors, including the hinge, the transmembrane domain, and the intracellular cell signaling domain, and methods for engineering and introducing such receptors into cells, are described, for example, in Chandran et al., “T cell receptor-based cancer immunotherapy: Emerging efficacy and pathways of resistance.” Immunological reviews 290.1 (2019): 127-147; Cartellieri, Marc, et al., “Chimeric antigen receptor-engineered T cells for immunotherapy of cancer.” BioMed Research International 2010 (2010); and PCT publication No. WO2017173256A1; US2002/131960, US2013/287748, US2013/0149337, U.S. Pat. Nos. 6,451,995, 7,446,190, 8,252,592; each of which is incorporated herein by reference in its entirety.

The present disclosure provides several CARs and CAR-expressing immune cells (e.g., NK cells) that can be used in immune cell therapies for treating cancer. In one aspect, provided herein are chimeric antigen receptors (CARs), wherein the CAR comprises: an extracellular antigen-binding domain; a hinge region; a transmembrane region; and an intracellular domain comprising a DAP10 intracellular signaling domain, a DAP12 intracellular signaling domain or DAP12, or a KIR2DS2 intracellular signaling domain.

The intracellular domain may comprise a DAP12 intracellular signaling domain. DNAX-activation protein 12 (DAP12) is a signaling adaptor molecule involved in signal transduction of activating NK cell receptors. In the immune system, DAP12 is found in cells of the myeloid lineage, such as macrophages and granulocytes, where it associates, for instance, with the triggering receptor expressed on myeloid cell members (TREM) and MDL1 (myeloid DAP12-associating lectin 1/CLEC5A), both involved in inflammatory responses against pathogens like viruses and bacteria. In the lymphoid lineage, DAP12 is expressed in NK cells and associates with activating receptors such as the C-type lectin receptor NKG2C, the natural cytotoxicity receptor NKp44, and the short-tailed KIR3DS1 and KIR2DS1/2/5, respectively. In particular, NGK2C is the dominant activating NK cell receptor for controlling CMV infection in both humans and mice.

The full-length amino acid sequence of DAP12 is known in the art (SEQ ID NO: 7). The intracellular domain may comprise the full-length sequence of DAP12 comprising an amino acid sequence of SEQ ID NO: 7 or an amino acid sequence that is about or at least 50%, 55%, 60%, 65%, 70%, 75%, 80%, 85%, 90%, 95%, 96%, 97%, 98%, or 99% identical to SEQ ID NO: 7.

The intracellular portion of the DAP12 is shown in SEQ ID NO: 32. The DAP12 intracellular signaling domain may comprise an intracellular portion of DAP12. The intracellular portion of DAP12 may comprise an amino acid sequence of SEQ ID NO: 32 or an amino acid sequence that is about or at least 50%, 55%, 60%, 65%, 70%, 75%, 80%, 85%, 90%, 95%, 96%, 97%, 98%, or 99% identical to SEQ ID NO: 32.

The intracellular domain may comprise a DAP10 intracellular signaling domain. The activating receptors expressed on natural killer cells recognize diverse ligands expressed on variety of malignant cells. Of them, natural killer group 2 D (NKG2D) is a type II membrane-oriented transmembrane receptor that is expressed on both NK cells and a majority proportion of CD8+ T cells. The membrane localization and signal transduction of NKG2D depend on DNAX-activating protein 10 (DAP10). DNAX-activating protein 10 (DAP10) interacts with natural killer group 2D (NKG2D), acting as an adaptor that targets various malignant cells for surveillance.

The intracellular portion of the DAP10 is shown in SEQ ID NO: 6. The DAP10 intracellular signaling domain may comprise an intracellular portion of DAP10. The intracellular portion of DAP10 may comprise an amino acid sequence of SEQ ID NO: 6 or an amino acid sequence that is about or at least 50%, 55%, 60%, 65%, 70%, 75%, 80%, 85%, 90%, or 95% identical to SEQ ID NO: 6.

The intracellular domain may further comprise an activation domain comprising a CD3ζ primary intracellular signaling domain. In immune cells, the CD3ζ homodimer transmits signals after binding of the low-affinity FcγRIII (CD16) to IgG-opsonized targets, thereby inducing Ab-dependent cellular cytotoxicity. The activation of CD3ζ-based CARs leads to an Ab-dependent cellular cytotoxicity-like activity of CAR-modified NK cells when engaging target cells.

The CD3ζ primary intracellular signaling domain may comprise an amino acid sequence of SEQ ID NO: 8 or an amino acid sequence that is about or at least 50%, 55%, 60%, 65%, 70%, 75%, 80%, 85%, 90%, or 95% identical to SEQ ID NO: 8.

The intracellular domain may comprise an intracellular signaling domain comprising a KIR2DS2 intracellular signaling domain. Killer cell immunoglobulin-like receptors (KIRs) are transmembrane glycoproteins expressed by natural killer cells and subsets of T cells. The KIR genes are polymorphic and highly homologous and they are found in a cluster on chromosome 19q13.4 within the 1 Mb leukocyte receptor complex (LRC). The gene content of the KIR gene cluster varies among haplotypes, although several “framework” genes are found in all haplotypes (KIR3DL3, KIR3DP1, KIR3DL4, KIR3DL2). The KIR proteins are classified by the number of extracellular immunoglobulin domains (2D or 3D) and by whether they have a long (L) or short (S) cytoplasmic domain. KIR proteins with the long cytoplasmic domain transduce inhibitory signals upon ligand binding via an immune tyrosine-based inhibitory motif (ITIM), while KIR proteins with the short cytoplasmic domain lack the ITIM motif and instead associate with the TYRO protein tyrosine kinase binding protein to transduce activating signals. The ligands for several KIR proteins are subsets of HLA class I molecules; thus, KIR proteins play an important role in regulation of the immune response. The intracellular signaling domain of KIR2DS2 is shown in SEQ ID NO: 11. The KIR2DS2 intracellular signaling domain may comprise an amino acid sequence of SEQ ID NO: 11 or an amino acid sequence that is about or at least 50%, 55%, 60%, 65%, 70%, 75%, 80%, 85%, 90%, or 95% identical to SEQ ID NO: 11.

The intracellular domain may further comprise a CD137 (4-1BB) co-stimulatory signaling domain. 4-1BB (TNFRSF9, CD137) is an activation-induced T cell costimulatory molecule. 4-1BB is usually expressed on a subset of resting CD8+ T cells and is upregulated on both CD4+ and CD8+ T cells following activation. Upon binding to trimeric 4-1BBL (TNFSF9, CD137L) on APCs, 4-1BB recruits TNFR-associated factor family members (TRAF1, TRAF2 and TRAF3) to its cytosolic region, forming the 4-1BB signalosome and leading to downstream activation of NF-κB, MAPK and ERK. The CD137 (4-1BB) co-stimulatory signaling domain may comprise an amino acid sequence of SEQ ID NO: 5 or an amino acid sequence that is about or at least 50%, 55%, 60%, 65%, 70%, 75%, 80%, 85%, 90%, or 95% identical to SEQ ID NO: 5.

The transmembrane region is a structural component, consisting of a hydrophobic alpha helix that spans the cell membrane. It anchors the CAR to the plasma membrane, bridging the extracellular hinge and antigen binding domains with the intracellular signaling region. This domain is essential for the stability of the receptor as a whole. Generally, the transmembrane domain from the most membrane-proximal component of the endodomain is used, but different transmembrane domains may result in different receptor stability. The transmembrane domain may be a transmembrane domain of CD137 (4-1BB), an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof. The transmembrane region may be a transmembrane region from CD8 (e.g., human CD8).

The transmembrane domain may be a CD8α transmembrane region. The amino acid sequence of the CD8α transmembrane region is shown in SEQ ID NO: 4. The CD8α transmembrane region may comprise an amino acid sequence of SEQ ID NO: 4 or an amino acid sequence that is about or at least 50%, 55%, 60%, 65%, 70%, 75%, 80%, 85%, 90%, or 95% identical to SEQ ID NO: 4.

The transmembrane region may be a KIR2DS2 transmembrane region. The amino acid sequence of the KIR2DS2 transmembrane region is shown in SEQ ID NO: 10. KIR2DS2 transmembrane region comprises an amino acid sequence of SEQ ID NO: 10 or an amino acid sequence that is about or at least 50%, 55%, 60%, 65%, 70%, 75%, 80%, 85%, 90%, or 95% identical to SEQ ID NO: 10.

The hinge, also called a spacer, is a small structural region that sits between the antigen binding domain and the cell's outer membrane. An ideal hinge enhances the flexibility of the scFv receptor head, reducing the spatial constraints between the CAR and its target antigen. This promotes antigen binding and synapse formation between the CAR-T cells and target cells. Hinge sequences are often based on membrane-proximal regions from immune molecules including e.g., IgG, CD8, and CD28.

The hinge region may be a membrane-proximal region from CD8, CD28, or any combination thereof. The hinge region may be a membrane-proximal region of CD8 (e.g., human CD8). The hinge region may be derived from an IgG (IgG1, IgG2, IgG3, or IgG4). The chimeric antigen receptors (CARs) or fragments thereof described herein may comprise a transmembrane region. The hinge region and the transmembrane region may be directly joined.

The hinge region may be a KIR2DS2 hinge region. The KIR2DS2 hinge region may comprise an amino acid sequence of SEQ ID NO: 9 or an amino acid sequence that is about or at least 50%, 55%, 60%, 65%, 70%, 75%, 80%, 85%, 90%, or 95% identical to SEQ ID NO: 9.

The hinge region may be a CD8α hinge region, the CD8α hinge region may comprise an amino acid sequence of SEQ ID NO: 3 or 41 or an amino acid sequence that is about or at least 50%, 55%, 60%, 65%, 70%, 75%, 80%, 85%, 90%, or 95% identical to SEQ ID NO: 3 or 41.

Provided herein can be a CAR comprising an extracellular antigen-binding domain; a CD8α hinge region; a CD8α transmembrane region; and an ICOS intracellular signaling domain, a CD3ζ primary intracellular signaling domain.

Provided herein can be a CAR comprising or consisting of an extracellular antigen-binding domain; a hinge region from CD8α; a transmembrane region from CD8α; and a CD27 intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a CD46 intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a DNAM intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a HVEM intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a 2B4 intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a 28H intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a 28H intracellular signaling domain, a 2B4 intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a 2B4 intracellular signaling domain, a 28H intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a CD3ζ primary intracellular signaling domain, a 2B4 intracellular signaling domain, and a 28H intracellular signaling domain.

Provided herein can be a CAR comprising or consisting of an extracellular antigen-binding domain; a hinge region from CD8α; a transmembrane region from CD8α; and a CD3ζ primary intracellular signaling domain, a 28H intracellular signaling domain, and a 2B4 intracellular signaling domain.

Provided herein can be a CAR comprising or consisting of an extracellular antigen-binding domain; a hinge region from CD8α; a transmembrane region from CD8α; and a CD137 (4-1BB) co-stimulatory signaling domain, a CD3ζ primary intracellular signaling domain, and a DAP10 intracellular signaling domain. The CAR may comprise an amino acid sequence of SEQ ID NO: 15 or an amino acid sequence that is about or at least 50%, 55%, 60%, 65%, 70%, 75%, 80%, 85%, 90%, or 95% identical to SEQ ID NO: 15. The CAR may comprise an amino acid sequence of SEQ ID NO: 33 or an amino acid sequence that is about or at least 50%, 55%, 60%, 65%, 70%, 75%, 80%, 85%, 90%, or 95% identical to SEQ ID NO: 33.

Provided herein can be a CAR comprising or consisting of an extracellular antigen-binding domain; a hinge region from CD8α; a transmembrane region from CD8α; and a CD137 (4-1BB) co-stimulatory signaling domain, a CD3ζ primary intracellular signaling domain, and a DAP12 intracellular signaling domain. The CAR may comprise an amino acid sequence of SEQ ID NO: 16 or an amino acid sequence that is about or at least 50%, 55%, 60%, 65%, 70%, 75%, 80%, 85%, 90%, or 95% identical to SEQ ID NO: 16. The CAR may comprise an amino acid sequence of SEQ ID NO: 34 or an amino acid sequence that is about or at least 50%, 55%, 60%, 65%, 70%, 75%, 80%, 85%, 90%, or 95% identical to SEQ ID NO: 34.

Provided herein can be a CAR comprising or consisting of an extracellular antigen-binding domain; a hinge region from CD8α; a transmembrane region from CD8α; and a KIR2DS2 intracellular signaling domain, and a CD3ζ primary intracellular signaling domain.

Provided herein can be a CAR comprising or consisting of an extracellular antigen-binding domain; a hinge region from CD8α; a transmembrane region from CD8α; and a KIR2DS2 intracellular signaling domain, a CD3ζ primary intracellular signaling domain; and a DAP12 intracellular signaling domain.

Provided herein can be a CAR comprising or consisting of an extracellular antigen-binding domain; a hinge region from KIR2DS2; a transmembrane region from KIR2DS2; and a KIR2DS2 intracellular signaling domain.

Provided herein can be a CAR comprising or consisting of an extracellular antigen-binding domain; a hinge region from KIR2DS2; a transmembrane region from KIR2DS2; and a KIR2DS2 intracellular signaling domain, and a DAP12 intracellular signaling domain or DAP12. The intracellular domain may comprise the entire DAP12 (i.e., full-length sequence of DAP 12). The CAR may comprise an amino acid sequence of SEQ ID NO: 17 or an amino acid sequence that is about or at least 50%, 55%, 60%, 65%, 70%, 75%, 80%, 85%, 90%, or 95% identical to SEQ ID NO: 17. The CAR may comprise an amino acid sequence of SEQ ID NO: 35 or an amino acid sequence that is about or at least 50%, 55%, 60%, 65%, 70%, 75%, 80%, 85%, 90%, or 95% identical to SEQ ID NO: 35.

Provided herein can be a CAR comprising or consisting of an extracellular antigen-binding domain; a hinge region from CD8α; a transmembrane region from CD8α; and a CD3ζ primary intracellular signaling domain, and a KIR2DS2 intracellular signaling domain. The CAR may comprise an amino acid sequence of SEQ ID NO: 18 or an amino acid sequence that is about or at least 50%, 55%, 60%, 65%, 70%, 75%, 80%, 85%, 90%, or 95% identical to SEQ ID NO: 18. The CAR may comprise an amino acid sequence of SEQ ID NO: 36 or an amino acid sequence that is about or at least 50%, 55%, 60%, 65%, 70%, 75%, 80%, 85%, 90%, or 95% identical to SEQ ID NO: 36.

Provided herein can be a CAR comprising or consisting of an extracellular antigen-binding domain; a hinge region from CD8α; a transmembrane region from CD8α; and a CD3ζ primary intracellular signaling domain, and a KIR2DS2 intracellular signaling domain, and a DAP12 intracellular signaling domain or DAP12. The intracellular domain may comprise the entire DAP12 (i.e., full-length sequence of DAP 12). The CAR may comprise an amino acid sequence of SEQ ID NO: 19 or an amino acid sequence that is about or at least 50%, 55%, 60%, 65%, 70%, 75%, 80%, 85%, 90%, or 95% identical to SEQ ID NO: 19. The CAR may comprise an amino acid sequence of SEQ ID NO: 37 or an amino acid sequence that is about or at least 50%, 55%, 60%, 65%, 70%, 75%, 80%, 85%, 90%, or 95% identical to SEQ ID NO: 37.

Provided herein can be a CAR comprising or consisting of an extracellular antigen-binding domain; a hinge region from CD8α; a transmembrane region from CD8α; and a CD137 (4-1BB) co-stimulatory signaling domain, and a CD3ζ primary intracellular signaling domain. The CAR may comprise an amino acid sequence of SEQ ID NO: 20 or an amino acid sequence that is about or at least 50%, 55%, 60%, 65%, 70%, 75%, 80%, 85%, 90%, or 95% identical to SEQ ID NO: 20.

Provided herein can be a CAR comprising or consisting of an extracellular antigen-binding domain; a hinge region from CD8α; a transmembrane region from CD8α; and a CD28 co-stimulatory signaling domain, and a CD3ζ primary intracellular signaling domain. The CAR may comprise an amino acid sequence of SEQ ID NO: 21 or an amino acid sequence that is about or at least 50%, 55%, 60%, 65%, 70%, 75%, 80%, 85%, 90%, or 95% identical to SEQ ID NO: 21.

Provided herein can be a CAR comprising or consisting of an extracellular antigen-binding domain; a hinge region from CD8α; a transmembrane region from CD8α; and a DAP12 intracellular signaling domain, a CD137 (4-1BB) co-stimulatory signaling domain, and a CD3ζ primary intracellular signaling domain. The CAR may comprise an amino acid sequence of SEQ ID NO: 22 or an amino acid sequence that is about or at least 50%, 55%, 60%, 65%, 70%, 75%, 80%, 85%, 90%, or 95% identical to SEQ ID NO: 22.

The elements in the CAR described herein are from the N-terminus to the C-terminus.

The CAR may comprise an amino acid sequence of any one of SEQ ID NOs: 15-19, and 22 or an amino acid sequence that is about or at least 50%, 55%, 60%, 65%, 70%, 75%, 80%, 85%, 90% or 95% identical to any one of SEQ ID NOs: 15-19 and 22.

The CAR described herein may comprise one of the following:
(i) an extracellular antigen-binding domain; a CD8α hinge region; a CD8α transmembrane region; and a CD137 (4-1BB) co-stimulatory signaling domain, a CD3ζ primary intracellular signaling domain, and a DAP10 intracellular signaling domain;
(ii) an extracellular antigen-binding domain; a CD8α hinge region; a CD8α transmembrane region; and a CD137 (4-1BB) co-stimulatory signaling domain, a CD3ζ primary intracellular signaling domain, and a DAP12 intracellular signaling domain;
(iii) an extracellular antigen-binding domain;
a KIR2DS2 hinge region; a KIR2DS2 transmembrane region; and a KIR2DS2 intracellular signaling domain;
(iv) an extracellular antigen-binding domain; a KIR2DS2 hinge region; a KIR2DS2 transmembrane region; and a KIR2DS2 intracellular signaling domain, and a DAP12 intracellular signaling domain;
(v) an extracellular antigen-binding domain;
a CD8α hinge region; a CD8α transmembrane region; and a CD3ζ primary intracellular signaling domain, and a KIR2DS2 intracellular signaling domain;
(vi) an extracellular antigen-binding domain; a CD8α hinge region; a CD8α transmembrane region; and a CD3ζ primary intracellular signaling domain, a KIR2DS2 intracellular signaling domain, and a DAP12 intracellular signaling domain;
and
(vii) an extracellular antigen-binding domain; a CD8α hinge region; a CD8α transmembrane region; and a CD3ζ primary intracellular signaling domain, and; a DAP12 intracellular signaling domain, CD137 (4-1BB) co-stimulatory signaling domain, and a CD3ζ primary intracellular signaling domain.

The CAR containing the DAP10 intracellular signaling domain or the DAP12 intracellular signaling domain located at the C-terminus of the intracellular structural domain have higher activity, efficacy and persistence. The order/placement of DAP12 in the CAR sequence is important in the activation and signaling of the CAR.

The elements in the CAR described herein are from the N-terminus to the C-terminus.

In one aspect, provided herein are chimeric antigen receptors (CARs), wherein the CAR comprises: an extracellular antigen-binding domain; a hinge region from CD8α or KIR2DS2; a transmembrane region from CD8α or KIR2DS2; and one or more intracellular signaling domain(s) comprising one or more protein(s) or a fragment thereof selected from CD137 (4-1BB), ICOS, CD3ζ, CD27, CD46, DNAM, HVEM, 2B4, 28H, DAP-10, DAP12, KIR2DS2, and CD28.

The extracellular antigen-binding domain of the CARs described herein can be any suitable antigen-binding fragments known in the art. The extracellular antigen-binding domain is exposed to the outside of the cell, in the ectodomain portion of the CAR. It interacts with potential target molecules and is responsible for targeting the CAR-expressing cell to any cell expressing a matching molecule. The antigen-binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. An scFv is a chimeric protein made up of the light (VL) and heavy (VH) chains of immunoglobulins, connected with a short linker peptide. The antigen binding domain may comprise one or more (e.g., 1, 2, 3, 4, 5, or 6) VH-VL pairs. The antigen binding domain may comprise one or more (e.g., 1, 2, 3, 4, 5, or 6) heavy chain single variable domains (V_{H}Hs). The V_{H}Hs may be connected with a linker peptide (e.g., a flexible linker). The linker peptide between the two V_{H}Hs includes hydrophilic residues with stretches of glycine and serine in it for flexibility as well as stretches of glutamate and lysine for added solubility.

The extracellular antigen-binding domain may be a Fab fragment, F(ab′) fragment, F(ab′)₂ fragment or Fv fragment. The antibody may be any one of various types of antibodies capable of binding antigen-specifically, briefly, having antigen-specific binding ability. For example, the antibody may be one in which one light chain and one heavy chain are bonded with each other, or one in which two light chains and two heavy chains are bonded with each other. For example, when two light chains and two heavy chains are bonded with each other, the antibody may be one in which a first unit including a first light chain and a first heavy chain bonded with each other and a second unit including a second light chain and a second heavy chain bonded with each other are combined with each other. The bond may be a disulfide bond. The two units may be the same as or different from each other. The first unit including the first light chain and the first heavy chain and the second unit including the second light chain and the second heavy chain may be the same as or different from each other. As such, an antibody prepared to recognize two different antigens by the first unit and the second unit, respectively, is typically referred to as a “bispecific antibody.” The antibody may be one in which three or more of the above-described units are combined with one another. The antigen-binding fragment of the present disclosure may be derived from various types of antibodies.

The linker peptide can comprise at least or about 5, 6, 7, 8, 9, 10, 11, 12, 13, 14, 15, 16, 17, 18, 19, 20, 30, 40, or 50 amino acid residues. The linker peptide may comprise at least or about 4, 5, 6, 7, 8, 9, 10, 11, 12, 13, 14, 15, 16, 20, 25, 30, or 40 glycine residues. The linker peptide may comprise at least or about 1, 2, 3, 4, 5, 6, 7, or 8 serine residues. The linker peptide may comprise or consist of both glycine and serine residues. The linker peptide may comprise or consist of a sequence that is at least or about 70%, at least or about 75%, at least or about 80%, at least or about 85%, at least or about 90%, at least or about 95%, at least or about 99%, or 100% identical to GGGGS (SEQ ID NO: 38) or GGGGSGGGGSGGGGS (SEQ ID NO: 39). The linker sequence may comprise at least 1, 2, 3, 4, 5, 6, 7, or 8 repeats of GGGGS (SEQ ID NO: 38). The linker sequence may have no more than 10, 11, 12, 13, 14, 15, 16, 17, 18, 19, 20, 30, 40, or 50 amino acid residues, the linker peptide may comprise 1, 2, 3, 4, or 5 amino acid insertions, deletions, or substitutions.

The extracellular antigen-binding domain may bind to a tumor antigen. Tumor antigens are proteins that are produced by tumor cells that elicit an immune response. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-1a, p53, prostein, PSMA, Her2/neu, survivin and telomerase, prostate carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The tumor antigen may include B-cell maturation antigen (BCMA), CD34, CD45, human leukocyte antigen-DR (HLA-DR), CD123, CD38, CLL1, CD105, CD71, SSC, MAGE, MUC16, CD 19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-13, IL-6, insulin-like growth factor I receptor, integrin a5b1, integrin anb3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-R1, TRAIL-R2, tumor antigen CTAA16.88, VEGF-A, VEGFR-1, VEGFR2, and vimentin, or viral-associated antigens expressed by the tumor.

The extracellular antigen-binding domain of the CAR may include an antigen binding moiety that when bound to its cognate antigen, affects a tumor cell such that the tumor cell fails to grow, or is promoted to die or diminish.

The extracellular antigen-binding domain of the CAR of described herein may target an antigen of a non-essential organ. The extracellular antigen-binding domain of the CAR described herein may include targets an antigen that is a biomarker for acute myeloid leukemia (AML). The biomarker for AML may include, but are limited to B-cell maturation antigen (BCMA), CD34, CD45, human leukocyte antigen-DR (HLA-DR), CD123, CD38, and CLL1.

Many CARs targeting different tumor antigens have been widely disclosed in the field, such as CD19 CARs or BCMA CARs. The extracellular antigen-binding domain of CD19 CARs can be or include the CD19 binding fragment (e.g., FMC63, SJ25C1, or those disclosed in different patents such as WO 2022/012683, etc). BCMA CARs also have been well described, related patents include but not limited to WO 2016/014789, WO 2016/014565, WO 2013/154760, and WO 2018/028647, etc.

The extracellular antigen-binding domain of BCMA (B-cell maturation antigen) CARs may be or include BCMA binding fragment. The BCMA binding fragment may bind to one or more epitopes on BCMA. The BCMA CARs may be bivalent CARs comprising two anti-BCMA sdAbs targeting different BCMA epitopes.

The extracellular antigen-binding domain may comprise two VHH domains targeting BCMA (see, e.g., WO 2018/028647, the entire content of which is incorporated herein). The extracellular antigen-binding domain may have an amino acid sequence of SEQ ID NO: 13 or a sequence that is about or at least 50%, 55%, 60%, 65%, 70%, 75%, 80%, 85%, 90%, or 95% identical to SEQ ID NO: 13.

The extracellular antigen-binding domain may include a first VHH antibody binding moiety comprising the amino acid sequence of SEQ ID NO: 23 or a sequence that is about or at least 50%, 55%, 60%, 65%, 70%, 75%, 80%, 85%, 90%, or 95% identical to SEQ ID NO: 23; and a second VHH antibody binding moiety comprising the amino acid sequence of SEQ ID NO: 24 or a sequence that is about or at least 50%, 55%, 60%, 65%, 70%, 75%, 80%, 85%, 90%, or 95% identical to SEQ ID NO: 24. CDR sequences can be determined according to well-known numbering systems. If not described to the contrary, CDRs are according to Kabat numbering. The CAR may comprise a first VHH antibody moiety that comprises a CDR1 comprising the amino acid sequence of SEQ ID NO: 25 or a sequence that is at least 80%, 85%, 90%, or 95% identical to SEQ ID NO: 25, a CDR2 comprising the amino acid sequence of SEQ ID NO: 26 or a sequence that is at least 80%, 85%, 90%, or 95% identical to SEQ ID NO: 26, and a CDR3 comprising the amino acid sequence of SEQ ID NO: 27 or a sequence that is at least 80%, 85%, 90%, or 95% identical to SEQ ID NO: 27, and a second VHH antibody moiety that comprises a CDR1 comprising the amino acid sequence of SEQ ID NO: 28 or a sequence that is at least 80%, 85%, 90%, or 95% identical to SEQ ID NO: 28, a CDR2 comprising the amino acid sequence of SEQ ID NO: 29 or a sequence that is at least 80%, 85%, 90%, or 95% identical to SEQ ID NO: 29, and a CDR3 comprising the amino acid sequence of SEQ ID NO: 30 or a sequence that is at least 80%, 85%, 90%, or 95% identical to SEQ ID NO: 30.

### Genetically Engineered Cells

The present disclosure provides engineered cells (e.g., immune cells, T cells, NK cells, tumor-infiltrating lymphocytes) that express the CAR described herein. These engineered cells can be used to treat various disorders or disease as described herein (e.g., a cancer).

The cell that is engineered may be obtained from, e.g., humans and non-human animals. The cell that is engineered may be obtained from bacteria, fungi, humans, rats, mice, rabbits, monkeys, pig or any other species. The cell may be from humans, rats or mice. The cells may be mouse lymphocytes and engineered (e.g., transduced) to express the CAR described herein. The cell may be obtained from humans. The cell that is engineered may be a blood cell. The cell may be a natural killer (NK) cell, T cell, a B cell, a macrophage/monocyte, peripheral blood mononuclear cell (PBMC), hematopoietic stem cell, pluripotent stem cell, and an embryonic stem cell. The cell may be NK cell. The cell may be T cell. The T cell may be a cytotoxic T cell, a helper T cell, a memory T cell, a Treg cell, a natural killer T (NK-T) cell, an up T cell, and a γδT cell.

Preparation of the engineered cells includes one or more culture and/or preparation steps. The cells for introduction of the binding molecule, e.g., CAR, can be isolated from a sample, such as a biological sample, e.g., one obtained from or derived from a subject. The subject from which the cell can be isolated is one having the disease or condition or in need of a cell therapy or to which cell therapy will be administered. The subject can be a human in need of a particular therapeutic intervention, such as the adoptive cell therapy for which cells are being isolated, processed, and/or engineered.

The cell may be an NK cell. Human natural killer cells (NK cells) play an important role in innate immune defense against malignant lymphoma cells, and thus are suitable for adoptive immune therapy (i.e., adoptive cellular immunotherapy). However, due to difficulties in ex vivo cell expansion and differences in activities of NK cells in individual subjects, it is difficult to use the NK cells.

NK cells are part of the innate immune system, providing the first line of defense against pathogens and cancer cells. They produce cytokines and mediate cytotoxicity without the need for prior sensitization and have the ability to interact with, and activate other immune cells. NK cells for immunotherapy can be generated from multiple sources, such as expanded autologous or allogeneic peripheral blood, umbilical cord blood, hematopoietic stem cells, induced pluripotent stem cells, as well as cell lines.

NK cells activation and effector function is a complex process as it depends upon the integration of signals from two distinct types of receptors-activating and inhibitory receptors. Normal healthy cells express MHC class I molecules on their surface, which act as ligands for inhibitory receptors and contribute to self-tolerance of NK cells. Cellular stress associated with viral infection or tumor development such as DNA damage, senescence or tumor suppressor genes upregulate ligands for activating receptors. This results in shift of balance to NK cells activation. Transmembrane and cytoplasmic stimulatory/activator molecules in NK cells can affect NK cell differentiation pathways, metabolic cycles, apoptosis as well as activation induced cell death.

The cell may be a T cell. The T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from subjects. Genetically modified T cells can be obtained by transducing T cells (e.g., isolated from the peripheral blood of subjects), with a viral vector. The T cells may be CD3+ T cells, CD8+ T cells, or regulatory T cells. The T cells may be T helper type 1 T cells and T helper type 2 T cells. The T cell expressing this receptor may be an αβT cell. The T cell expressing this receptor may be a γδT cell. The T cells may be central memory T cells. The T cells may be effector memory T cells. The T cells may be naïve T cells.

The cells may be stem cells, such as multipotent and pluripotent stem cells, including induced pluripotent stem cells (iPSCs). The cells can be primary cells, such as those isolated directly from a subject and/or isolated from a subject and frozen. The stem cells may be cultured with additional differentiation factors to obtain desired cell types (e.g., NK cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. Any known method for separation based on such markers can be used. The separation may be affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 8%, 90%, 91%, 92%, 93%, 94%, 95%, 96%, 97%, 98%, 99%, or more percent of the total cells in the composition or cells of a certain type such as NK cells, T cells, CD8+ or CD4+ cells.

The engineered cells (e.g. CAR-NK cells) may be co-cultured with target cells (e.g., antigen presenting cells) for at least or about 1 hour, 2 hours, 3 hours, 4 hours, 5 hours, 6 hours, 12 hours, 18 hours, 1 day, 2 days, 3 days, or longer, such that the engineered cells (e.g., CAR-NK cells) can be activated.

The engineered cells (e.g., CAR-NK cells) may express an armor protein. The armor protein may be secreted IL-15 (e.g., wild type IL-15, preprotein IL-15 or mutant IL-15). The CAR may be fused with secreted IL-15 by furin P2A. The furin P2A may comprise an amino acid sequence of SEQ ID NO: 12 or a portion thereof. The CAR may be fused with secreted IL-15 by P2A, the P2A may comprise an amino acid sequence of SEQ ID NO: 40 or a portion thereof.

The engineered cells (e.g., CAR-NK cells) may have a cytotoxicity effect on tumor cells. The engineered cells (e.g., CAR-NK cells) may kill about 10%, about 20%, about 30%, about 40%, about 50%, about 60%, about 70%, about 80%, about 90% or more tumor cells when contacted with the tumor cells. The engineered cells (e.g., CAR-NK cells) may be contacted with tumor cells at a ration of about 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, or 2:1.

The engineered cells (e.g., CAR-NK cells) may be contacted with the tumor cells for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or more.

The engineered cells (e.g., CAR-NK cells) may retain the CAR after contacted with the tumor cells. The engineered cells (e.g., CAR-NK cells) may retain about 5%, 10%, 15%, 20%, 25%, 30%, 35%, 40%, 45%, 50%, 55%, 60%, 65%, 70%, 75%, 80%, 85%, 90%, 95% or more of the CAR expressed on the cells after contacted with the target cells.

The engineered cells (e.g., CAR-NK cells) may induce the production of one or more cytokines after contacted with the target cells. The cytokine may be interferon γ (IFN-γ). The cytokine may be granulocyte-macrophage colony-stimulating factor (GM-CSF).

The engineered cells (e.g., CAR-NK cells) may reduce or slow down tumor progression in a subject having cancer (e.g., BCMA positive multiple myeloma). The progression of the cancer may be evaluated every 1, 2, 3, 4, 5, 6, or 7 days. The progression of the cancer may be evaluated every 1, 2, 3, 4 weeks. The progression of the cancer may be evaluated every 1, 2, 3, 4, 5, 6, 7, 8, 9, 10, 11, 12 or more months. The engineered cells (e.g., CAR-NK cells) may retain therapeutic activity, efficacy and persistence after about 1, 2, 3, 4, 5, 6, or 7 days. The engineered cells (e.g., CAR-NK cells) may retain therapeutic activity, efficacy and persistence after about 1, 2, 3, 4 weeks. The engineered cells (e.g., CAR-NK cells) may retain therapeutic activity, efficacy and persistence after about 1, 2, 3, 4, 5, 6, 7, 8, 9, 10, 11, 12 or more months. About 5%, 10%, 15%, 20%, 25%, 30%, 35%, 40%, 45%, 50%, 55%, 60%, 65%, 70%, 75%, 80%, 85%, 90%, 95% or more of the engineered cells (e.g., CAR-NK cells) may be retained after about 1, 2, 3, 4, 5, 6, or 7 days, about 1, 2, 3, 4 weeks, or about 1, 2, 3, 4, 5, 6, 7, 8, 9, 10, 11, 12 or more months.

The engineered cells (e.g., CAR-NK cells) are able to proliferate or expand when contacted with the target cells. The engineered cells (e.g., CAR-NK cells) are able to proliferate or expand for about 1 fold, about 2 folds, about 3 folds, about 4 folds, about 5 folds, about 6 folds, about 7 folds, about 8 folds, about 9 folds, about 10 folds, about 11 folds, about 12 folds, about 13 folds, about 14 folds, about 15 folds or more during a time period of about 5 to about 100 hours.

### Nucleic Acids and Recombinant Vectors

The present disclosure also provides recombinant vectors (e.g., an expression vectors) that include a nucleic acid or a set of nucleic acids disclosed herein (e.g., a polynucleotide that encodes a CAR disclosed herein), host cells into which are introduced the recombinant vectors (i.e., such that the host cells contain the CAR and/or a vector comprising the CAR), and the production of CARs by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

The vector may be a viral vector. The viral vector can be grown in a culture medium specific for viral vector manufacturing. Any suitable growth media and/or supplements for growing viral vectors can be used in accordance with the embodiments described herein.

The vector used may be a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

The vector or construct can contain a single promoter that drives the expression of one or more nucleic acid molecules. Such promoters can be multicistronic (bicistronic or tricistronic). For example, transcription units can be engineered as a bicistronic unit containing an IRES (internal ribosome entry site), which allows coexpression of gene products (e.g. encoding CAR and an antibody or antigen binding fragment thereof) by a message from a single promoter. Alternatively, in some cases, a single promoter may direct expression of an RNA that contains, in a single open reading frame (ORF), two or three genes (e.g. encoding CAR and/or an antibody or antigen binding fragment thereof) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Provided herein are vectors encoding CARs or fragments thereof. The vectors may comprise a nucleic acid sequence that encodes any one of the CARs described herein. The vectors may encode an amino acid sequence that is about or at least 50%, 55%, 60%, 65%, 70%, 75%, 80%, 85%, 90%, or 95% identical to SEQ ID NOs: 15-19 and 22. Sequence of the vectors can be codon-optimized.

The vector may encode a fusion polypeptide comprising a CAR described herein (e.g., the CAR comprising an amino acid sequence that is about or at least 50%, 55%, 60%, 65%, 70%, 75%, 80%, 85%, 90%, or 95% identical to SEQ ID NOS: 15-19 and 22) and an armor molecule. The armor protein may be secreted IL-15 (e.g., wild type IL-15, preprotein IL-15 or mutant IL-15). The CAR may be fused with secreted IL-15 by furin P2A. The furin P2A may comprise an amino acid sequence of SEQ ID NO: 12 or a portion thereof. The CAR may be fused with secreted IL-15 by P2A. The P2A may comprise an amino acid sequence of SEQ ID NO: 40 or a portion thereof.

The present disclosure also provides a nucleic acid sequence comprising a nucleotide sequence encoding any of the CARs, antigen binding fragments thereof, and/or CAR-derived binding molecules (including, e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. The nucleotide sequence can be codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

The CAR, or antigen-binding fragment thereof is encoded by a nucleotide sequence that has been codon-optimized. The polypeptide may comprise a signal peptide. The signal peptide may be a GM-CSF signal peptide comprising an amino acid sequence of SEQ ID NO: 1. The signal peptide may be a CD8α signal peptide comprising an amino acid sequence of SEQ ID NO: 2. The polypeptide and/or the fusion protein may be recombinant.

The disclosure also provides a nucleic acid sequence that is at least 1%, 2%, 3%, 4%, 5%, 6%, 7%, 8%, 9%, 10, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. The disclosure relates to nucleotide sequences encoding any peptides that are described herein, or any amino acid sequences that are encoded by any nucleotide sequences as described herein.

The nucleic acid sequence may be at least or about 10, 20, 30, 40, 50, 60, 70, 80, 90, 100, 110, 120, 130, 150, 200, 250, 300, 350, 400, 500, or 600 nucleotides. The amino acid sequence may be at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. The nucleic acid sequence may be less than 10, 20, 30, 40, 50, 60, 70, 80, 90, 100, 110, 120, 130, 150, 200, 250, 300, 350, 400, 500, or 600 nucleotides. The amino acid sequence may be less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Methods for Preparation of Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. The subject from which the cell is isolated may be one having the disease or condition or in need of a cell therapy or to which cell therapy will be administered. The subject may be a human in need of a particular therapeutic intervention, such as the adoptive cell therapy for which cells are being isolated, processed, and/or engineered.

Accordingly, the cells can be primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

The cells can be derived from cell lines, e.g., T cell lines. The cells can be obtained from a xenogeneic source, for example, from mouse, rat, or non-human primate. The cells can be isolated from mouse lymph nodes.

The blood cells collected from the subject may be washed, e.g., to remove the plasma fraction and to place the cells in an appropriate buffer or media for subsequent processing steps. The cells may be washed with phosphate buffered saline (PBS). The wash solution may lack calcium and/or magnesium and/or many or all divalent cations. A washing step may be accomplished a semi-automated “flow-through” centrifuge. A washing step may be accomplished by tangential flow filtration (TFF). The cells may be resuspended in a variety of biocompatible buffers after washing, such as, for example, Ca²⁺/Mg²⁺ free PBS. Components of a blood cell sample may be removed and the cells directly resuspended in culture media. The methods may include density-based cell separation methods, such as the preparation of white blood cells from peripheral blood by lysing the red blood cells and centrifugation through a Percoll or Ficoll gradient.

The method may comprise one or more steps of: e.g., isolating the T cells or NK cells from a subject's blood; transducing the population T cells or NK cells with a viral vector including the nucleic acid construct encoding a genetically engineered antigen receptor; expanding the transduced cells in vitro; and/or infusing the expanded cells into the subject, where the engineered T cells or NK cells will seek and destroy antigen positive tumor cells. The nucleic acid construct may further include a sequence encoding an inhibitory protein. The method further may comprise transfection of T cells or NK cells with the viral vector containing the nucleic acid construct.

The methods involve introducing any vectors described herein into a cell in vitro or ex vivo. The vector may be a viral vector and the introducing is carried out by transduction. The cell may be transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer.

The transfection of T cells or NK cells can be achieved by using any standard method such as calcium phosphate, electroporation, liposomal mediated transfer, microinjection, biolistic particle delivery system, or any other known methods by skilled artisan. Transfection of T cells may be performed using the calcium phosphate method.

The present disclosure provides a method to create a personalized anti-tumor immunotherapy. Genetically engineered T cells or NK cells can be produced from a subject's blood cells. These engineered T cells are then reinfused into the subject as a cellular therapy product.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. The treatment can halt, slow, retard, or inhibit progression of a cancer. The treatment can result in the reduction of in the number, severity, and/or duration of one or more symptoms of the cancer in a subject.

In one aspect, the disclosure features methods that include administering a therapeutically effective amount of engineered cells expressing CAR (e.g., a CAR-NK), antigen binding fragments thereof, to a subject in need thereof (e.g., a subject having, or identified or diagnosed as having, a cancer).

The subject may have cancer. The cancer may be acute lymphoblastic leukemia (AML); acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; lung cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

The compositions and methods disclosed herein can be used for treatment of subjects at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a subject or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, subject history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some aspects, the present disclosure also provides methods of diagnosing a disease/condition in a mammal, wherein the CARs interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer or infection. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has infection or cancer.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered cells and compositions thereof to subjects, e.g., subjects or animal models (e.g., mice).

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. The composition may include at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for subject. Additionally, a preparation which is in such form as to permit the biological activity of an active ingredient contained therein to be effective, and which contains no additional components which are unacceptably toxic to a subject to which the formulation would be administered.

The choice of carrier may be determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. A mixture of two or more preservatives can be used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. A mixture of two or more buffering agents can be used. The buffering agent ormixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/orvincristine.

The pharmaceutical composition may contain the cells in amounts effective to treat or prevent the disease or condition, such as a therapeutically effective or prophylactically effective amount. Therapeutic or prophylactic efficacy can be monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. The cell populations can be administered parenterally. The term “parenteral,” as used herein, includes intravenous, intramuscular, subcutaneous, rectal, vaginal, and intraperitoneal administration. The cells can be administered to the subject using peripheral systemic delivery by intravenous, intraperitoneal, or subcutaneous injection.

The compositions can be provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added.

Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtrationmembranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. The methods described herein can reduce the risk of the developing diseases, conditions, and disorders as described herein.

The cells, populations, and compositions, described herein can be administered to a subject or subject having a particular disease or condition to be treated, e.g., via adoptive cell therapy, such as CAR-NK cell therapy. Cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered CAR-NK cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, “Cell transfer immunotherapy for metastatic solid cancer what clinicians need to know.” Nature reviews Clinical oncology 8.10 (2011): 577; Themeli et al., “Generation of tumor-targeted human T lymphocytes from induced pluripotent stem cells for cancer therapy.” Nature biotechnology 31.10 (2013): 928; Tsukahara et al., “CD19 target-engineered T cells accumulate at tumor lesions in human B-cell lymphoma xenograft mouse models.” Biochemical and biophysical research communications 438.1 (2013): 84-89; Davila et al., “CD19 CAR-targeted T cells induce long-term remission and B Cell Aplasia in an immunocompetent mouse model of B cell acute lymphoblastic leukemia.” PloS one 8.4 (2013); each of which is incorporated herein by reference in its entirety.

The cell therapy, e.g., CAR-NK cell therapy, can be carried out by autologous transfer, in which the T cells are isolated and/or otherwise prepared from the subject who is to receive the cell therapy, or from a sample derived from such a subject. Thus, in some aspects, the cells are derived from a subject, e.g., subject, in need of a treatment and the cells, following isolation and processing are administered to the same subject.

The cell therapy, e.g., CAR-NK cell therapy, can be carried out by allogeneic transfer, in which the T cells are isolated and/or otherwise prepared from a subject other than a subject who is to receive or who ultimately receives the cell therapy, e.g., a first subject. The cells then can be administered to a different subject, e.g., a second subject, of the same species. The first and second subjects may be genetically identical. The first and second subjects may be genetically similar. The second subject may express the same HLA class or supertype as the first subject.

The subject may have been treated with a therapeutic agent targeting the disease or condition, e.g. the tumor, prior to administration of the cells or composition containing the cells. The subject may be refractory or non-responsive to the other therapeutic agent. The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The cells can be administered at a desired dosage, which in some aspects includes a desired dose or number of cells or cell type(s) and/or a desired ratio of cell types. Thus, the dosage of cells may be based on a total number of cells (or number per kg body weight) and a desired ratio of the individual populations or sub-types, such as the CD4+ to CD8+ ratio. The dosage of cells may be based on a desired total number (or number per kg of body weight) of cells in the individual populations or of individual cell types. The dosage may be based on a combination of such features, such as a desired number of total cells, desired ratio, and desired total number of cells in the individual populations.

The populations or sub-types of cells, such as NK cells, can be administered at or within a tolerated difference of a desired dose of total cells, such as a desired dose of NK cells. The desired dose may be a desired number of cells or a desired number of cells per unit of body weight of the subject to whom the cells are administered, e.g., cells/kg. The desired dose may be at or above a minimum number of cells or minimum number of cells per unit of body weight.

The desired dose may be a desired number of cells of the sub-type or population, or a desired number of such cells per unit of body weight of the subject to whom the cells are administered, e.g., cells/kg. The desired dose may be at or above a minimum number of cells of the population or sub-type, or minimum number of cells of the population or sub-type per unit of body weight.

Thus, the dosage may be based on a desired fixed dose of total cells and a desired ratio, and/or based on a desired fixed dose of one or more, e.g., each, of the individual sub-types or sub-populations.

The cells or individual populations of sub-types of cells, can be administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

The dose of total cells and/or dose of individual sub-populations of cells may be within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, the cells can be administered at, or within a certain range of error of, between at or about 10⁴ and at or about 10⁹ NK cells/kilograms (kg) body weight, such as between 10⁵ and 10⁶ NK cells/kg body weight, for example, at least or at least about or at or about 1×10⁵ NK cells/kg, 1.5×10⁵ NK cells/kg, 2×10⁵ NK cells/kg, or 1×10⁶ NK cells/kg body weight.

Optimal response to therapy can depend on the ability of the engineered recombinant receptors such as CARs, to be consistently and reliably expressed on the surface of the cells and/or bind the target antigen. For example, in some cases, properties of certain recombinant receptors, e.g., CARs, can affect the expression and/or activity of the recombinant receptor, in some cases when expressed in a cell, such as a human T cell, used in cell therapy. In some contexts, the level of expression of particular recombinant receptors, e.g., CARs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. The desired ratio (E:T ratio) may be between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. The E:T ratio may be greater than or about 1:1, 2:1, 3:1, 4:1, 5:1, 6:1, 7:1, 8:1, 9:1, or 10:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells may be suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. They can be administered by parenteral, intrapulmonary, and intranasal, and, if desired for local treatment, intralesional administration. Parenteral infusions include intramuscular, intravenous, intraarterial, intraperitoneal, or subcutaneous administration. A given dose can be administered by a single bolus administration of the cells. It can be administered by multiple bolus administrations of the cells, or by continuous infusion administration of the cells.

### EXAMPLES

The invention is further described in the following examples, which do not limit the scope of the invention described in the claims.

### Example 1: Preparation of CAR-NK Cells Targeting BCMA

### Construction of Chimeric Antigen Receptors (CARs)

Various CAR constructs were designed with NK cell specific domains. We utilized NK cells specific signaling domains such as DAP10 (SEQ ID NO: 6), DAP12 (SEQ ID NO: 7), KIR2DS2 (SEQ ID NO: 11), besides CD137 (4-1BB SEQ ID NO: 5) specific constructs as control. Designs of constructs are shown in Table 1. Constructs 32 and 34 contain intracellular portion of DAP12 (SEQ ID NO: 32, underlined in SEQ ID NO: 7), while construct 36 and 38 contain full length DAP12 sequence. Construct 36 and 38 contain KIR2DS2 followed by furin P2A, full length DAP 12 sequence. All CAR sequences are connected with secreted wild type IL15 (SEQ ID NO: 31, underlined sequence in SEQ ID NO: 14) by furin P2A except BBz and CD28z construct. BBz and CD28z constructs contain the prepro IL15 connected by P2A (SEQ ID NO: 40, underlined sequence in SEQ ID NO: 12).

TABLE 1
NK Specific CAR Constructs Design

| | | | | Intracellular | | |
|---|---|---|---|---|---|---|
| | | | | domain (ICD) | | |
| | Extracellular antigen | | Transmembrane | (From the N-terminus | | |
| CAR code | binding domain | Hinge | (TM) | to the C-terminus) | Connector | Armor |
| 20 | BCMA VHH1-VHH2 | CD8α | CD8α | ICOS CD3ζ | Furin P2A | IL15 |
| 21 | BCMA VHH1-VHH2 | CD8α | CD8α | CD27 CD3ζ | Furin P2A | IL15 |
| 22 | BCMA VHH1-VHH2 | CD8α | CD8α | CD46 CD3ζ | Furin P2A | IL15 |
| 23 | BCMA VHH1-VHH2 | CD8α | CD8α | DNAM CD3ζ | Furin P2A | IL15 |
| 24 | BCMA VHH1-VHH2 | CD8α | CD8α | HVEM CD3ζ | Furin P2A | IL15 |
| 25 | BCMA VHH1-VHH2 | CD8α | CD8α | 2B4 CD3ζ | Furin P2A | IL15 |
| 26 | BCMA VHH1-VHH2 | CD8α | CD8α | 28H CD3ζ | Furin P2A | IL15 |
| 27 | BCMA VHH1-VHH2 | CD8α | CD8α | 28H 2B4 CD3ζ | Furin P2A | IL15 |
| 28 | BCMA VHH1-VHH2 | CD8α | CD8α | 2B4 28H CD3ζ | Furin P2A | IL15 |
| 29 | BCMA VHH1-VHH2 | CD8α | CD8α | CD3ζ 2B4 28H | Furin P2A | IL15 |
| 30 | BCMA VHH1-VHH2 | CD8α | CD8α | CD3ζ 28H 2B4 | Furin P2A | IL15 |
| 31 | BCMA VHH1-VHH2 | CD8α | CD8α | CD137 CD3ζ DAP10 | Furin P2A | IL15 |
| 32 | BCMA VHH1-VHH2 | CD8α | CD8α | CD137 CD3ζ DAP12 | Furin P2A | IL15 |
| 33 | BCMA VHH1-VHH2 | CD8α | CD8α | KIR2DS2 CD3ζ | Furin P2A | IL15 |
| 34 | BCMA VHH1-VHH2 | CD8α | CD8α | KIR2DS2 CD3ζ DAP12 | Furin P2A | IL15 |
| 35 | BCMA VHH1-VHH2 | KIR2DS2 | KIR2DS2 | KIR2DS2 | Furin P2A | IL15 |
| 36 | BCMA VHH1-VHH2 | KIR2DS2 | KIR2DS2 | KIR2DS2 DAP12 | Furin P2A | IL15 |
| 37 | BCMA VHH1-VHH2 | CD8α | CD8α | CD3ζ KIR2DS2 | Furin P2A | IL15 |
| 38 | BCMA VHH1-VHH2 | CD8α | CD8α | CD3ζ KIR2DS2 DAP12 | Furin P2A | IL15 |
| BBZ | BCMA VHH1-VHH2 | CD8α | CD8α | CD137 CD3ζ | P2A | Prepro |
| | | | | | | IL15 |
| CD28Z | BCMA VHH1-VHH2 | CD8α | CD8α | CD28 CD3ζ | P2A | Prepro |
| | | | | | | IL15 |

### Construction of CAR-Natural Killer (NK) Cells

### NK Expansion

CAR-NK cells were cultured with PBMC as the starting material. CD3 depleted PBMC cells were cultured in a medium containing cytokine cocktail including IL2. Cells were transduced with retrovirus (generated from various retrovirus constructs containing CAR designs in Table1) using spinocculation (MOI 1, 2000g, 32 °C). Cells were harvested on day 18. Functional potential of cells was measured by various assays including proliferation assay, serial cytotoxicity assay, cytokine secretion assay against NCI-H929 tumor cells (ATCC Cat No: CRL9068).

### Example 2: In Vitro Screening of CAR-NK Cells

### In Vitro Cytotoxicity of CAR-NK Cells

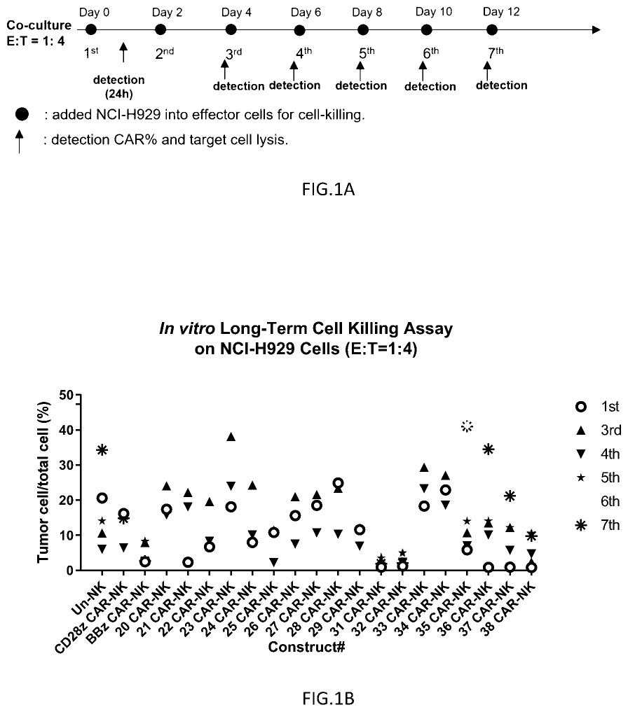

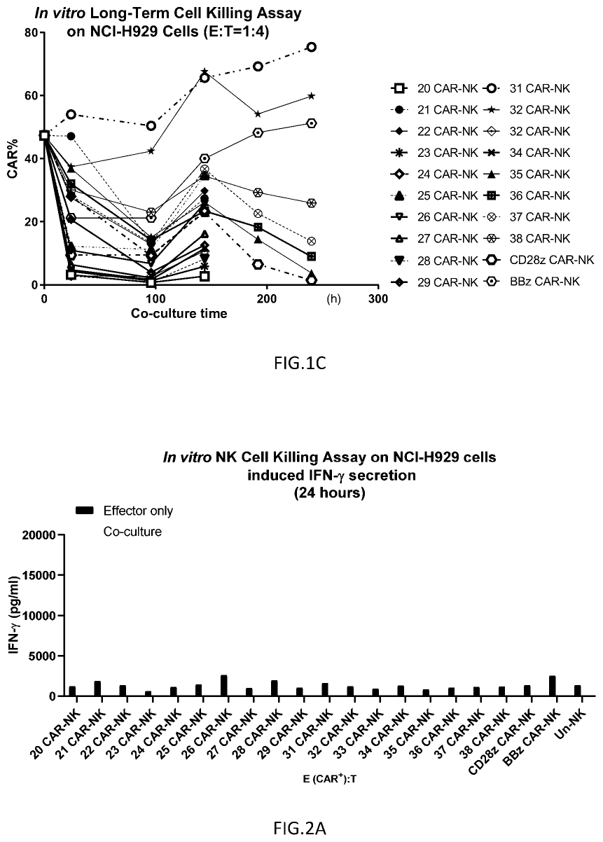

Designed constructs were evaluated for their cytotoxicity potential in a serial cytotoxicity assay. CAR-NK and control NK cells were washed in assay medium (KBM+5% HS), counted and adjusted to a density of 1 million per mL. Similarly, freshly labeled (CellTrace Violet, Thermofisher Cat No C34557) tumor cells (NCI-H929) were washed and adjusted to a density of 1 million per mL. CAR-NK cells were co-cultured with labeled tumor cells at specific E/T 1:1 ratio in a 12 well plate. Freshly labeled tumor cells were added every 48 hours in the initial co-culture of CAR-NK cells and NCI-H929 cells. Analysis of repetitive killing of tumor (NCI-H929) cells by CAR-NK and control cells was performed at various time points with flowcytometer (BD FACSCelesta™ Cell Analyzer). As shown in FIG. 1B and FIG. 1C, CAR-NK cells expressing constructs 31, 32, 35, 36, 37 and 38 were superior in long term serial killing assay. CAR-NK cells expressing construct 31 and CAR-NK cells expressing construct 32 were able to kill NCI H929 cells more effectively in vitro. Additionally, we stained cells for CAR detection using FITC conjugated BCMA protein (Acrobiosystems Cat No BCA-HF254) at various time points. CAR-NK cells expressing construct 31 or 32 retained higher percentage of CAR positive NK cells than control BBz CAR-NK cells.

### Detection of IFNγ and GM-CSF Release Level in CAR-NK Cells

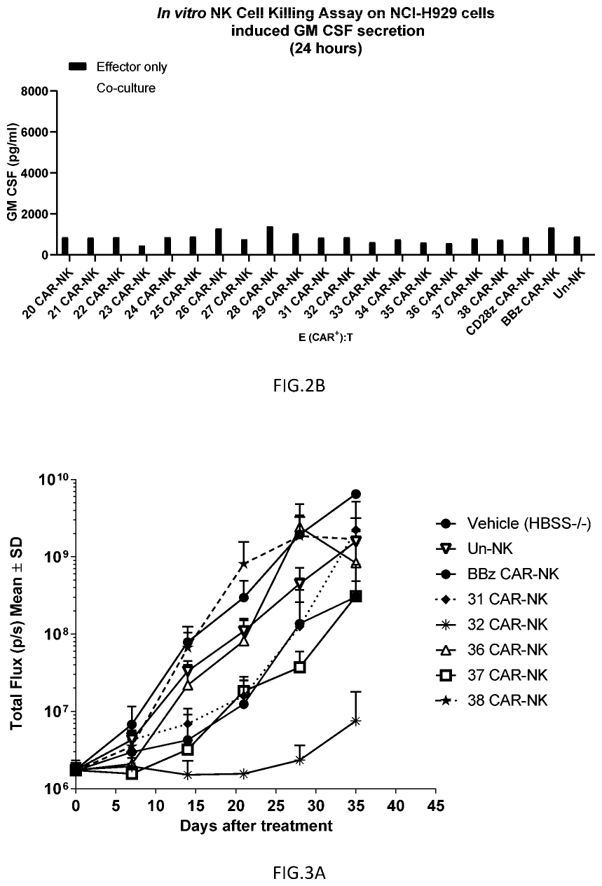

We also evaluated the cytokine release potential of CAR-NK cells. We investigated IFNγ as well as GM-CSF levels. We performed ELISA for IFNγ (Cisbio, Cat #62HIFNGPEH) and GM-CSF (Cisbio, Cat #62HGMCSFPEH) from medium/soup collected from co-cultures of CAR-NK and NCI-H929 cells at E/T 1:1 ratio for 24 h. Briefly, we prepared appropriate dilutions of samples and standards. Further, we added 16 μl dilutions of standard protein or samples per well in 96 well plates. We then added 4 μl of premixed antibodies for either IFNγ or GM-CSF in respective wells. Plates were incubated overnight and in HTRF compatible spectrophotometer (BMG Labtech, PHERAstar® FSX HTS). Indeed, as shown in FIGS. 2A and 2B, CAR-NK cells expressing construct 31 or 32 secreted higher or comparable amount of IFNγ at 24 h compared to control. In addition, CAR-NK cells expressing construct 31 or 32 secreted higher GM-CSF than others at 24 h post co-culture. Secretion of inflammatory cytokine IFNγ correlate with higher activity in long-term killing assay.

### Example 3: In Vivo Evaluation of Selected CAR Design Constructs

We then investigated the functional potential against NCI-H929 cells in vivo of selected CAR constructs including 31 (SEQ ID NO: 15), 32 (SEQ ID NO: 16), 36 (SEQ ID NO: 17), 37 (SEQ ID NO: 18) and 38 (SEQ ID NO: 19). We utilized disseminated tumor mouse model in NCG mice (NOD/ShiLtJGpt-Prkdc^{em26ca52}Il2rg^{em26ca22}/Gpt). Briefly, to create the tumor xenograft, NCG mice were injected intravenously with NCI-H929-Luc cells (1×10⁶ cells/mouse, BCMA positive multiple myeloma cell line, #ATCC CRL-9068™, transduced with Luciferase). Nine days later, tumor engrafted mice were treated with sIL-15 wt armored CAR-NK cells (4 mice per group), or un-transduced NK cells comprising human IL-15 at 0.5 μg/mouse (i.e., hIL-15 intraperitoneal injection or “UnNK cell, i.v., IL-15, i.p.”).

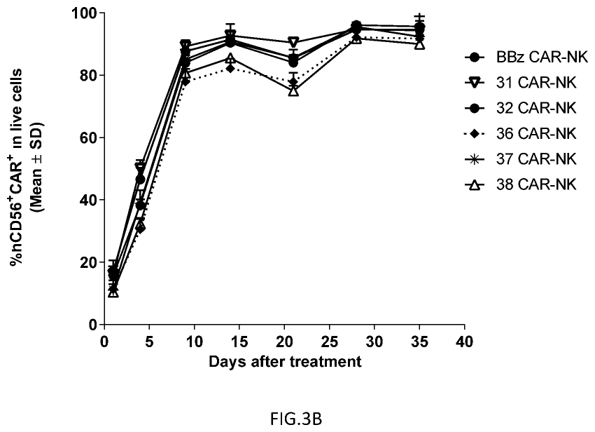

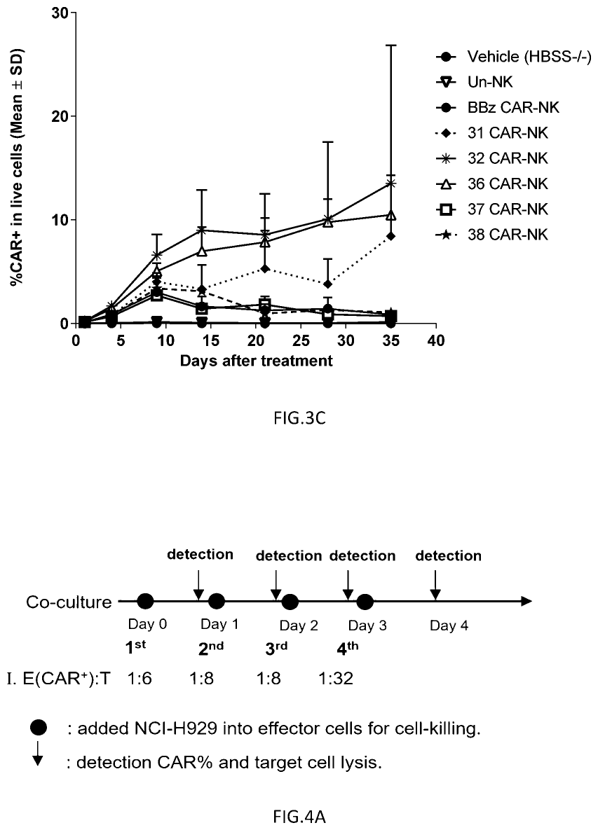

Mice were infused with 2×10⁶ CAR⁺ NK cells at day 0, 2, and 1×10⁶ CAR⁺ NK cells at day 5 respectively to a total dose of 5×10⁶ CAR*NK cells. Tumor progression was evaluated by in vivo bioluminescence imaging (BLI, PerkinElmer IVIS® Lumina LT In Vivo Imaging System) weekly at each time point. Indeed, as shown in FIG. 3A, construct 32 based BCMA CAR-NK cells had highest tumor clearing activity in vivo. We also collected mice blood at various time points and measured number of human NK and CAR-NK cells using immunostaining. CAR-NK cells expressing construct 32 also had comparable cell numbers/persistence along with CAR-NK cells expressing construct 31 or 37 in FIG. 3B. CAR-NK cells expressing construct 32 showed higher number of total CAR positive cells as shown in FIG. 3C. The above results demonstrate that CAR-NK cells expressing construct 32 retained highest activity, efficacy and persistence.

### Example 4: Evaluation of CAR Constructs Containing DAP12

Since construct 32 contains NK specific DAP12 as the signaling molecule. We further investigated whether different positions of DAP12 affected functional activity of CAR-NK cells. Therefore, we designed additional constructs as depicted Table 2. Each CAR sequence is connected with secreted mutated IL15 (mutIL15) by P2A. MutIL15 is a secreted mutated IL15 comprising an amino acid substitution at position 62 as defined by SEQ ID NO: 31 (IL-15 polypeptide) (see, e.g., PCT Application No. WO2023280307A1, the entire content of which is incorporated herein).

TABLE 2
NK Specific CAR Constructs with DAP12

| | | | | Intracellular | | |
|---|---|---|---|---|---|---|
| | | | | signaling domain (ICD) | | |
| CAR code | Extracellular antigen | | Transmembrane | (From the N-terminus | | |
| (SEQ ID NO.) | binding domain | Hinge | (TM) | to the C-terminus) | Connector | Armor |
| 352M (SEQ | BCMA VHH1-VHH2 | CD8α | CD8α | DAP12 CD137 CD3ζ | P2A | mutIL15 |
| ID NO: 22) | | | | | | |
| 32M | BCMA VHH1-VHH2 | CD8α | CD8α | CD137 CD3ζ DAP12 | P2A | mutIL15 |
| BBzM | BCMA VHH1-VHH2 | CD8α | CD8α | CD137 CD3ζ | P2A | mutIL15 |

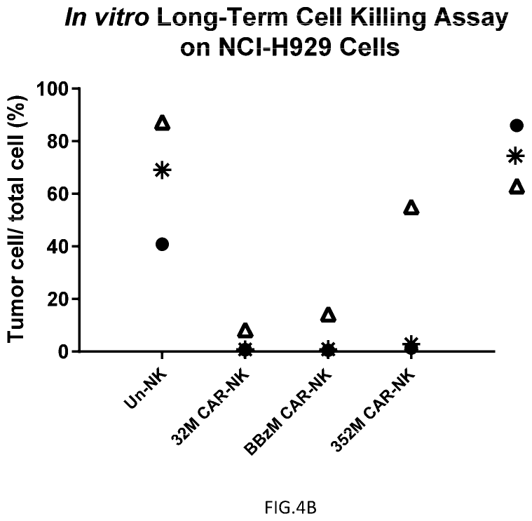

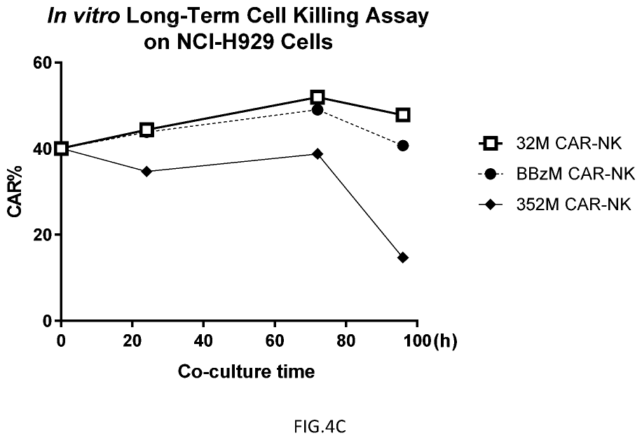

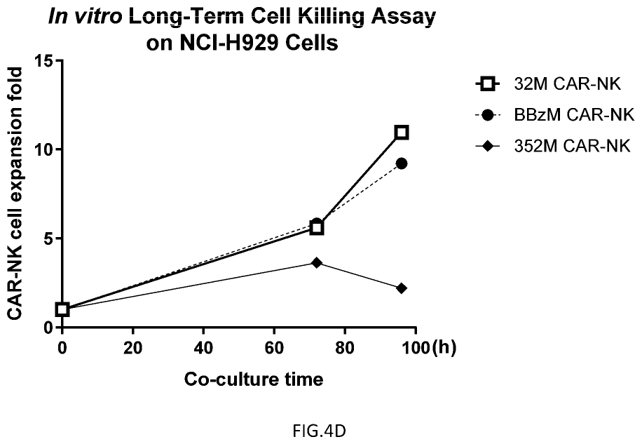

We then compared the efficacy of new constructs with DAP12 at different positions. We performed serial cytotoxicity assay as described above. Serial cytotoxicity assay results showed that 32M CAR-NK cells retained activity after four rounds of tumor exposure, while BBzM CAR-NK cells and 352M CAR-NK cells lost their activity as shown in FIG. 4B. We also analyzed percentage CAR and found similar result suggesting that 32M CAR-NK cells retained CAR expression until four rounds of tumor exposure followed by BBzM CAR-NK cells and 352M CAR-NK cells, as shown in FIG. 4C.

We also counted NK and CAR-NK cells to analyze proliferation or expansion during co-culture experiment at various time points. Proliferation results during different rounds of tumor exposure also showed that 32M CAR-NK cells proliferate better than BBzM CAR-NK cells, which proliferate better than 352M CAR-NK cells (FIG. 4D). These results indicate that 32M CAR is the most active construct, suggesting that DAP12 is better placed at the end of the CAR construct (the C-terminus of the CAR construct) than at the beginning.

In conclusion, we showed that the order/placement of DAP12 in the CAR sequence is equally important as the selection of signaling molecules. Despite using domains from multiple stimulatory molecules, we observed that CD137 domain in this context has been very crucial. Our construct 32 also utilized CD3ζ as well as DAP12, indicating that multiple signal relay molecules could be beneficial for NK-specific CAR. Overall, our study demonstrates the importance of DAP12 and provides directions for new design of CAR-NK cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chimeric antigen receptor (CAR), wherein the CAR comprises:
an extracellular antigen-binding domain;
a transmembrane region; and
an intracellular domain comprising a DAP10 intracellular signaling domain, a DAP12 intracellular signaling domain or DAP12, or a KIR2DS2 intracellular signaling domain.

2. The CAR of claim 1, wherein the intracellular domain further comprises an activation domain comprising a CD3ζ primary intracellular signaling domain.

3. The CAR of claim 1 or 2, wherein the DAP10 intracellular signaling domain, the DAP12 intracellular signaling domain, or the KIR2DS2 intracellular signaling domain is located at the C-terminus of the intracellular domain.

4. The CAR of any one of claims 1-3, further comprising a co-stimulatory signaling domain located between the C-terminus of the transmembrane region and the N-terminus of the CD3ζ primary intracellular signaling domain.

5. The CAR of claim 4, wherein the co-stimulatory signaling domain is CD137 (4-1BB) co-stimulatory signaling domain.

6. The CAR of any one of claims 1-3, wherein the intracellular domain comprises a KIR2DS2 intracellular signaling domain.

7. The CAR of claim 6, wherein the CAR further comprises a DAP12 at the C-terminus of the intracellular signaling domain.

8. The CAR of any one of claims 1-7, wherein the transmembrane region is a transmembrane region derived from: α chain of a T cell receptor, β chain of the T cell receptor, ζ chain of the T cell receptor, CD8α, CD28, CD3ε, CD3δ, CD3γ, CD33, CD37, CD64, CD80, CD45, CD4, CD5, CD8, CD9, CD16, CD22, CD86, CD137 or CD154.

9. The CAR of any one of claims 1-8, wherein the transmembrane region is a transmembrane region derived from CD8α.

10. The CAR of any one of claims 1-9, further comprising a hinge domain located between the C-terminus of the extracellular antigen binding domain and the N-terminus of the transmembrane domain.

11. The CAR of any one of claims 1-10, wherein the hinge region is a hinge region derived from: CD8α, CD28, IgG1, IgG2, IgG3, or IgG4.

12. The CAR of any one of claims 1-11, wherein the hinge region is a hinge region derived from CD8α.

13. The CAR of any one of claims 1-5, wherein the CAR comprises from the N-terminus to the C-terminus as following: an extracellular antigen-binding domain; a CD8α hinge region; a CD8α transmembrane region; and a CD137 (4-1BB) co-stimulatory signaling domain, a CD3ζ primary intracellular signaling domain, and a DAP12 intracellular signaling domain.

14. The CAR of claim 1, wherein the transmembrane region is a transmembrane region derived from KIR2DS2.

15. The CAR of claim 14, further comprising a hinge domain located between the C-terminus of the extracellular antigen binding domain and the N-terminus of the transmembrane domain, wherein the hinge is a hinge region derived from KIR2DS2.

16. The CAR of claim 14 or 15, wherein the intracellular domain comprises a KIR2DS2 intracellular signaling domain.

17. The CAR of claim 16, wherein the CAR further comprises a DAP12 at the C-terminus of the intracellular signaling domain.

18. The CAR of any one of claims 1-17, wherein the extracellular antigen-binding domain comprises a scFv, a (scFv)2, a VHH domain, or a VNAR domain.

19. The CAR of claim 18, wherein the extracellular antigen-binding domain comprises one or more VHH domains.

20. The CAR of the any one of claims 1-19, wherein the extracellular antigen-binding domain binds to a tumor antigen.

21. The CAR of claim 20, wherein the tumor antigen is selected from the group consisting of: B-cell maturation antigen (BCMA), CD34, CD45, human leukocyte antigen-DR (HLA-DR), CD123, CD38, CLL1, CD105, CD71, SSC, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125, CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-13, IL-6, insulin-like growth factor I receptor, integrin a5b1, integrin anb3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-R1, TRAIL-R2, tumor antigen CTAA16.88, VEGF-A, VEGFR-1, VEGFR2, and vimentin.

22. The CAR of claim 21, wherein the extracellular antigen-binding domain binds to BCMA.

23. The CAR of claim 22, wherein the CAR comprises a first VHH antibody moiety that comprises a CDR1 comprising the amino acid sequence of SEQ ID NO: 25, a CDR2 comprising the amino acid sequence of SEQ ID NO: 26, and a CDR3 comprising the amino acid sequence of SEQ ID NO: 27, and a second VHH antibody moiety that comprises a CDR1 comprising the amino acid sequence of SEQ ID NO: 28, a CDR2 comprising the amino acid sequence of SEQ ID NO: 29, and a CDR3 comprising the amino acid sequence of SEQ ID NO: 30.

24. The CAR of claim 22 or 23, wherein the CAR comprises a first VHH antibody moiety comprising the amino acid sequences of SEQ ID NO: 23 or an amino acid sequence having at least 90%, 95%, or 99% identity to the amino acid sequence of SEQ ID NO: 23, and a second VHH antibody moiety comprising the amino acid sequences of SEQ ID NO: 24 or an amino acid sequence having at least 90%, 95%, or 99% identity to the amino acid sequence of SEQ ID NO: 24.

25. The CAR of any one of claims 22-24, wherein the CAR comprises an amino acid sequence set forth in SEQ ID NO: 13, or an amino acid sequence having at least 90%, 95%, or 99% identity to the amino acid sequence set forth in any one of SEQ ID NO: 13.

26. The CAR of claim 5, wherein the CAR comprises an amino acid sequence of any one of SEQ ID NOs: 15-16 or an amino acid sequence that is at least 80%, 85%, 90% or 95% identical to any one of SEQ ID NOs: 15-16.

27. The CAR of claim 6, wherein the CAR comprises an amino acid sequence of SEQ ID NO: 18 or an amino acid sequence that is at least 80%, 85%, 90% or 95% identical to SEQ ID NO: 18.

28. The CAR of claim 7, wherein the CAR comprises an amino acid sequence of SEQ ID NO: 19 or an amino acid sequence that is at least 80%, 85%, 90% or 95% identical to SEQ ID NO: 19.

29. The CAR of claim 15, wherein the CAR comprises an amino acid sequence of SEQ ID NO: 17 or an amino acid sequence that is at least 80%, 85%, 90% or 95% identical to SEQ ID NO: 17.

30. A nucleic acid comprising a nucleotide sequence encoding the CAR of any one of claims 1-29.

31. A modified immune cell comprising the nucleic acid of claim 30.

32. The modified immune cell of claim 31, wherein the modified immune cell is selected from the group consisting of: a natural killer (NK) cell, T cell, a B cell, a macrophage/monocyte, peripheral blood mononuclear cell (PBMC), hematopoietic stem cell, pluripotent stem cell, and an embryonic stem cell.

33. The modified immune cell of claim 32, wherein the cell is NK cell.

34. The modified immune cell of any one of claims 31-33, wherein the cell further comprises a nucleotide sequence encoding an IL-15 polypeptide.

35. The modified immune cell of claim 34, wherein the IL-15 polypeptide comprises an amino acid substitution at position 62.

36. The modified immune cell of claim 35, wherein the amino acid substitution at position 62 is T62G.

37. The modified immune cell of claim 36, wherein the IL-15 polypeptide comprises an amino acid sequence having at least 90% sequence identity to the amino acid sequence of SEQ ID NO: 31.

38. A pharmaceutical composition comprising the modified immune cell of any one of claims 31-37, and a pharmaceutical acceptable carrier.

39. A method for treating and/or preventing cancer comprising administering an effective amount of the modified immune cell of any one of claims 31-37 or pharmaceutical composition of claim 38 to a subject in need thereof.
